(19) United States
(12) Patent Application Publication
(10) Pub. No.: US 2025/0207175 A1
(43) Pub. Date: Jun. 26, 2025

(54) METHOD FOR DETECTING CONTAMINATING CARBOXYLESTERASE ACTIVITY

(71) Applicant: Boehringer Ingelheim International GmbH, Ingelheim am Rhein (DE)

(72) Inventors: Oliver BURKERT, Mittelbiberach (DE); Sebastian WELZ, Ulm (DE); Matthias KNAPE, Warthausen (DE)

(21) Appl. No.: 18/844,130

(22) PCT Filed: Mar. 8, 2023
(86) PCT No.: PCT/EP2023/055876

§ 371 Date: Sep. 5, 2024

(30) Foreign Application Priority Data
Mar. 9, 2022 (EP) 22161135.3

(51) Int. Cl. C12Q 1/44; G01N 33/58

(52) U.S. Cl. CPC C12Q 1/44; G01N 33/582; G01N 2333/918

(57) ABSTRACT

The present invention relates to a method for detecting carboxylesterase activity of contaminating host cell protein in a sample comprising a recombinant protein of interest produced in a eukaryotic cell in cell culture comprising contacting the sample with the hydrophilic substrate HPTS ester and optionally in addition separately contacting the sample with a lipophilic substrate 4-MU ester and detecting the carboxylesterase and optionally lipase activity of the at least one contaminating host cell protein using the hydrophilic and optionally lipophilic substrate by detecting the fluorescence intensity of the released chromophore. Further provided is a method for manufacturing a recombinant protein of interest comprising using the method for detecting carboxylase activity of a contaminating host cell protein in a sample comprising a recombinant protein of interest produced in a eukaryotic cell in cell culture using a hydrophilic substrate and optionally further a lipophilic substrate for determining contaminating carboxylesterase and/or lipase activity in the sample comprising the recombinant protein of interest during manufacture.

### FIELD OF THE INVENTION

The present invention relates to a method for detecting carboxylesterase activity of contaminating host cell protein in a sample comprising a recombinant protein of interest produced in a eukaryotic cell in cell culture comprising contacting the sample with the hydrophilic substrate HPTS ester and optionally in addition separately contacting the sample with a lipophilic substrate 4-MU ester and detecting the carboxylesterase and optionally lipase activity of the at least one contaminating host cell protein using the hydrophilic and optionally lipophilic substrate by detecting the fluorescence intensity of the released chromophore. Further provided is a method for manufacturing a recombinant protein of interest comprising using the method for detecting carboxylase activity of a contaminating host cell protein in a sample comprising a recombinant protein of interest produced in a eukaryotic cell in cell culture using a hydrophilic substrate and optionally further a lipophilic substrate for determining contaminating carboxylesterase and/or lipase activity in the sample comprising the recombinant protein of interest during manufacture.

### BACKGROUND

Proteins as therapeutic agents have become increasingly popular in the last decades. Formulations comprising therapeutic proteins, such as monoclonal antibodies, often contain high protein concentration of 100 mg/ml or higher and often require the presence of a detergent. The most widely used detergents in biopharmaceutical industry due to their biocompatibility and low toxicity are polysorbates (PS), such as polysorbate 20 (polyoxyethylene (20) sorbitan monolaurate, Tween 206) or polysorbate 80 (polyoxyethylene (20) sorbitan monooleate, Tween 806).

Polysorbates are heterogeneous mixtures of sorbitol and its anhydrides along with approximately 20 polymerized ethylene oxide moieties partially esterified with fatty acids. However, polysorbates are prone to degradation, which can adversely affect product quality. Degradation may affect product quality not only due to the resulting reduced polysorbate concentration in the formulation, but also due to the formation of visible and sub-visible particles from insoluble matter of polysorbate degradants, such as fatty acids and polyoxyethylene side chains. Polysorbates can be degraded chemically or enzymatically. Chemical polysorbate degradation is mainly caused by an oxidative reaction causing the formation of inter alia aldehydes, ketones and fatty acids. Enzymatic polysorbate degradation is characterized by hydrolysis of the ester bond connecting the polyethoxylated sorbitan with the fatty acid (Dwivedi et al., 2018, International Journal of Pharmaceutics 552:442-436). Although oxidative degradation of polysorbates has been known for a long time, enzymatic hydrolysis of polysorbates in antibody formulations have only recently been considered as one of the major degradation pathways. In the recent years, polysorbate degradation has emerged as a major challenge in the biopharmaceutical community.

It has been reported that residual host cell proteins (HCPs) with hydrolytic activity, such as lipases and other esterases, including carboxylesterases, in the final drug product (DP) can lead to polysorbate degradation. The identified enzymes were mainly assigned to the class of lipases, a subclass of esterases catalyzing the hydrolysis of lipids. The role of lipases in the degradation of polysorbates in antibody formulations has further been emphasized by Chiu et al., wherein harvested cell culture fluid (HCCF) from lipoprotein lipase (LPL) knockout CHO cells reduced the PS20 and PS80 degradation as compared to wild type CHO cells (Chui et al., 2017, Biotechnol. Bioeng. 114, 1006-1015). Also group XV lysosomal phospholipase A2 isomer X1 (LPLA2), putative phospholipase B-like-2 (PLBL2), liver carboxylesterase, phospholipase A2 group VII (PLA2G7), lysophospholipase 2 (LYPLA2), lysosomal acid lipase (LIPA), sialic acid acetylesterase (SIAE), palmitoyl-protein thioesterase 1 (PPT1) and lipoprotein lipase (LPL) were detected in activity-based protein profiling assays and final drug products indicating these enzymes as potentially critical towards PS degradation (Hall et al., 2016, Journal of Pharmaceutical Sciences, 105(5): 1633-1642; Dixit et al., 2016, Journal of Pharmaceutical Sciences, 105(5): 1657-1666; Zhang et al., 2020, Journal of Pharmaceutical Sciences, 109(11): 3300-3307; Chiu et al., 2017, Biotechnology and bioengineering 114(5): 1006-1015; Li et al., 2020, Anal. Chem., 93(23): 8161-8169; Graf et al., 2021, Journal of Pharmaceutical Sciences, 110:3558-3567). Yet, some of these enzymes belong to the class of esterases that favour more hydrophilic substrates, such as carboxylesterases. Necessary alterations and adaptations of the upstream and particularly downstream production processes in therapeutic protein production are difficult, as determining the impact of single purification steps and conditions on polysorbate degradation takes several weeks.

Polysorbate content and degradation can be studied using different analytical techniques. The most commonly used method for quantification of polysorbates is reverse phase liquid chromatography (such as RP-HPLC) and this may further be coupled to evaporative light scattering detector (ELSD) and charged aerosol detector (CAD). Other techniques capable of polysorbate content determination consists of fluorescence micelle assay (FMA) or a chemical complexation of the sorbitan ring with cobalt thiocyanate or ferric thiocyanate. However, in order to determine whether alterations in the purification process were successful in terms of reducing the hydrolytic activity responsible for polysorbate degradation, samples of interest need to be spiked with polysorbate and its degradation needs to be analyzed as described above. Thus, polysorbate degradation is typically assessed by monitoring the decrease of polysorbate content over time. However, polysorbate degradation is a slow process that may take up to several weeks or months. Further, the analytics are complex and time consuming.

In order to develop purification conditions that minimize enzymatic polysorbate degradation in the drug product, there is a need for a fast, reliable automated high-throughput assay with high sensitivity, which can be easily adapted to the different samples and provides predictive information about hydrolytic activity responsible for polysorbate degradation in a drug substance of drug product sample. Such an assay is useful as tool to guide process development for drug substance production with increased product quality due to minimized polysorbate degrading activity co-purified with the target protein.

Detection of hydrolytic activity of lipases and carboxylesterases in vitro using fluorescent substrates has been known in the art, but these prior art assays are not sufficiently sensitive to reliably detect within a short period of time contaminating lipase activity in a recombinant protein preparation, which has only been co-purified from eukaryotic cells with the recombinant protein (protein of interest). For example, Tsuzuki et al., (Biosci. Biotechnol. Biochem, 2001, 65(9): 2078-2082) analyse the activity of several lipases from microorganisms at high concentrations using fluorescent substrates. Likewise, Yoo et al., (Cell Chemical Biology, 2020, 27: 143-157) discloses a fluorogenic substrate assay for detecting lipase activity and uses Triton X-100 for solubilizing the highly concentrated lipase rPfMAGLLP prior to analysis. WO 2010/024924 discloses an assay for screening for lipases expressed in E. coli using a fluorogenic substrate. A fluorimetric assay for detecting activity of hydrolases, including carboxylesterases has further been reported by Wolfbeis and Koller (Analytical Biochemistry, 1983, 129: 365-370), but again only isolated and concentrated hydrolases have been tested. Yet, none of these assays is described therein to be used for detecting lipase activity of a contaminating host cell protein in a sample comprising a recombinant protein purified from eukaryotic cells.

Menden et al., 2019 (Journal of Enzyme Inhibition of Medicinal Chemistry, 34(1): 1474-1480) reported a lipase activity assay in which lipase activity of a defined enzyme extract of Candida rugosa lipase (CRL) isoforms is detected to verify the mode of action of the inhibitor tropolone using 4-methylumbelliferyl butyrate (4-MUB) and palmitate (4-MUP) as substrate. Limitations to the assay are reported, which include the intrinsic decrease in solubility of the hydrophobic fatty acid tail with length and the autocatalysis of the substrate in the basic pH range. Moreover, no detergent is used in the assay. More recently, Jahn et al., 2020 (Pharm. Res. 37(118): 2-13) reports a chromophore-based lipase activity assay for use in determining polysorbate degradation in samples of harvested cell culture fluids using 4-methylumbelliferyl oleate (4-MuO) as substrate. However, the moderate sensitivity still requires incubation times of 24 hour or more. 4-methylumbelliferyl-based substrates for detecting lipase activity have further been described by Bhargava et al., 2021 (Pharm Res. 38(3): 397-413), WO 2022/047415 A1 and in WO 2022/049294 A1. These recent efforts illustrate the need for assays to detect polysorbate degrading activity during biopharmaceutical development. Yet, all these assays focus on lipophilic substrates and lipase activity using conditions that favor activity of certain polysorbate degrading enzymes and hence other polysorbate degrading activities may be missed. Also, Bhargava et al., 2021 (Pharm Res. 38(3): 397-413) and WO 2022/047415 A1 address as imitations to the disclosed esterase activity assay that residual HCPs may differ in their hydrolytic activities towards the carboxylic ester bonds in MU-C8 versus PS20 or PS80 and that the pH 8.0 used is not representative for the low pH used in parenteral drug products.

Accordingly, there is a need for a fast and high throughput method to broaden the spectrum and supplement the existing assays for the detection of polysorbate degrading enzymes, such as carboxylesterases, with high sensitivity.

### SUMMARY OF THE INVENTION

The present invention relates to a method for detecting carboxylesterase activity of contaminating host cell protein in a sample comprising a recombinant protein of interest produced in a eukaryotic cell comprising (a) providing at least one sample comprising a recombinant protein of interest produced in a eukaryotic cell in cell culture and at least one contaminating host cell protein; (b) contacting the at least one sample with a reaction solution (comprising a hydrophilic substrate) to form a reaction mixture, wherein the reaction solution comprises: (i) a buffer having a pH of about pH 4 to about pH 8, (ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and (iii) optionally a non-buffering salt; (c) incubating the sample and the substrate in the reaction mixture; and (d) detecting carboxylesterase activity of the at least one contaminating host cell protein by measuring hydrolysis of the substrate HPTS ester and detecting the fluorescence intensity of the released chromophore 1-hydroxypyrene-3,6,8-trisulfonic acid or a salt thereof (HPTS); optionally measuring hydrolysis by detecting the fluorescence intensity of the released chromophore HPTS over time, while incubating the sample and the substrate in the reaction mixture according to step (c). In certain embodiments the sample and the substrate in the reaction mixture are incubated for any time period between 2 min and 5 hours, 2 min and 3 hours, 2 min and 2 hours, or 2 min and 0.5 hours; and/or the reaction mixture has a volume of 300 μl or less. According to the method, multiple reaction mixtures may be analysed in parallel, preferably in a volume of 300 μl or less.

The fluorescence of the released chromophore HPTS is determined preferably using an excitation wavelength within a range of 401-405 nm and an emission wavelength within a range of 510-516 nm. In preferred embodiments, the substrate HPTS ester is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof. In a more preferred embodiment, the substrate HPTS ester is selected from the group consisting of 1-octanoyloxy-pyrene-3,6,8-trisulfonic acid trisodium salt (OPTS), 1-nonaoyloxy-pyrene-3,6,8-trisulfonic acid trisodium salt and 1-decanoyloxy-pyrene-3,6,8-trisulfonic acid trisodium salt. Preferably the substrate HPTS ester is used at a final concentration in the reaction mixture of 50 μM or less or preferably 30 μM or less.

In preferred embodiments, the method further comprises (bi) contacting the at least one sample comprising a recombinant protein of interest produced in a eukaryotic cell in cell culture and at least one contaminating host cell protein of step (a) in a separate reaction set-up with a reaction solution comprising a lipophilic substrate to form a reaction mixture, wherein the reaction solution comprises: (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester, (iii) optionally a non-buffering salt, and (iv) a non-denaturing detergent not having an ester-bond, wherein the detergent is a non-ionic or zwitterionic detergent, (ci) incubating the sample and the substrate in the reaction mixture of step (bi); and (di) detecting lipase activity of the at least one contaminating host cell protein by measuring hydrolysis of the substrate 4-MU ester and detecting the fluorescence intensity of the released chromophore 4-MU; optionally measuring hydrolysis by detecting the fluorescence intensity of the released chromophore 4-MU overtime, while incubating the sample and the substrate in the reaction mixture according to step (ci).

The lipophilic substrate is preferably selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate. Micelle formation improves lipase activity and solubilization of the lipophilic substrate. Thus, according to the invention the detergent used in the reaction solution comprising the lipophilic substrate has a final concentration in the reaction mixture above its critical micelle concentration in the reaction mixture. In certain embodiments, the detergent is selected from the group consisting of CHAPS, CHAPSO and Zwittergent, preferably CHAPS. In case the detergent is CHAPS, it is preferably provided at a final concentration in the reaction mixture of about 8 mM to about 20 mM, preferably at about 8 mM to about 15 mM, more preferably at about 10 mM. In certain embodiments the detergent is not polyethylene glycol tert-octylphenyl ether (Triton X-100) and not polyethylene glycol nonylphenyl ether (NP-40).

The buffer used in the method of the invention (using the hydrophilic and/or lipophilic substrate) may comprises one or more buffer substances selected from the group consisting of a formic acid, acetic acid, lactic acid, citric acid, malic acid, maleic acid, glycine, glycylglycine, succinic acid, TES (2-{[tris(hydroxyme-thyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄. In certain embodiments, the buffer has a pH of about 5 to about 7.5, preferably the buffer has a pH of about 5.5 to about 7.0. The buffer may also be a multi-component buffer having a buffering range from at least about pH 5 to at least about pH 7.5, preferably from at least about pH 4 to at least about pH 8.

The optionally non-buffering salt may be selected from the group consisting of NaCl, KCl and CaCl₂, preferably the non-buffering salt is NaCl or KCl. Moreover, the non-buffering salt may have a concentration of about 100 mM to about 200 mM, preferably about 130 mM to about 170 mM, more preferably about 140 mM to about 150 mM in the reaction mixture. In certain embodiments, the ionic strength of the non-buffering salt is about 200 mM or less in the reaction mixture, preferably about 150 mM or less in the reaction mixture; and/or the cumulative ionic strength of the buffer and the non-buffering salt in the reaction mixture is about 450 mM or less, preferably about 400 mM or less, more preferably about 350 mM or less in the reaction mixture.

The at least one sample according to certain embodiments is a harvested cell culture fluid (HCCF), an in-process control (IPC) sample, a UF/DF filtrate, a drug substance sample or a drug product sample. In preferred embodiments, the recombinant protein of interest is produced in a CHO cell and the at least one contaminating host cell protein is a CHO host cell protein (CHOP). According to the invention the recombinant protein of interest is not a carboxylesterase or a lipase and/or an enzyme having carboxylesterase or lipase activity. Preferably, the recombinant protein of interest is selected from the group consisting of an antibody, an antibody fragment, an antibody derived molecule and a fusion protein.

The present invention further provides a method of manufacturing a recombinant protein of interest comprising the steps of (i) cultivating a eukaryotic cell expressing a recombinant protein of interest in cell culture; (ii) harvesting the recombinant protein of interest; (iii) purifying the recombinant protein of interest; and (iv) optionally formulating the recombinant protein of interest into a pharmaceutically acceptable formulation suitable for administration; and (v) obtaining at least one sample comprising the recombinant protein of interest in steps (ii), (iii) and/or (iv); wherein the method further comprises detecting carboxylesterase activity in a sample comprising the recombinant protein of interest and at least one contaminating host cell protein comprising: (a) providing the at least one sample obtained in step (v) comprising the recombinant protein of interest produced in a eukaryotic cell in cell culture and at least one contaminating host cell protein; (b) contacting the at least one sample with a reaction solution comprising a hydrophilic substrate to form a reaction mixture, wherein the reaction solution comprises: (i) a buffer having a pH of about pH 4 to about pH 8, (ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and (iv) optionally a non-buffering salt; (c) incubating the sample and the substrate in the reaction mixture; and (d) detecting carboxylesterase activity of the at least one contaminating host cell protein by measuring hydrolysis of the substrate HPTS ester and detecting the fluorescence intensity of the released chromophore HPTS; wherein the method optionally further comprises detecting lipase activity in a sample comprising (bi) contacting the at least one sample comprising a recombinant protein of interest produced in a eukaryotic cell and at least one contaminating host cell protein of step (a) in a separate reaction set-up with a reaction solution comprising a lipophilic substrate to form a reaction mixture, wherein the reaction solution comprises; (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester, (iii) optionally a non-buffering salt, and (iv) a non-denaturing detergent not having an ester-bond, wherein the detergent is a non-ionic or zwitterionic detergent, (ci) incubating the sample and the substrate in the reaction mixture; (di) detecting lipase activity of the at least one contaminating host cell protein by measuring hydrolysis of the 4-MU ester and detecting the fluorescence intensity of the released chromophore 4-MU; optionally measuring hydrolysis by detecting the fluorescence intensity of the released chromophore HPTS and/or 4-MU over time, while incubating the sample and the substrate in the reaction mixture according to step (c) or (ci), respectively.

The method for manufacture according to the invention may further comprise obtaining at least one sample comprising the recombinant protein of interest in step (ii), wherein the sample is a harvested cell culture fluid (HCCF) or a cell lysate; in step (iii), wherein the sample is an in-process control (IPC) sample; and/or in step (iv), wherein the sample is a UF/DF sample, a drug substance sample or a drug product sample. In a preferred embodiment, the method comprises obtaining at least one sample comprising the recombinant protein of interest produced in a eukaryotic cell in cell culture and at least one contaminating host cell protein in step (iii), comprising obtaining at least one sample before and after affinity chromatography, before and after acid treatment, before and after depth filtration, and/or before and after ion exchange chromatography, preferably anion exchange chromatography or cation exchange chromatography.

In yet another aspect, the invention relates to a kit for determining contaminating carboxylesterase and/or lipase activity in a sample comprising a recombinant protein of interest comprising: (i) a buffer having a pH of about pH 4 to about pH 8; and (ii) a hydrophilic substrate and a lipophilic substrate, wherein (a) the hydrophilic substrate is a saturated unbranched-chain fatty acid (C6-C12) ester of 1-hydroxypyrene-3,6,8-trisulfonic acid or a salt thereof (substrate HPTS ester); and (b) the lipophilic substrate comprises the chromophore 4-methylumbelliferyl (4-MU) in the form of a 4-MU ester, wherein the 4-MU ester is a saturated unbranched-chain fatty acid (C6-C16) 4-MU ester (substrate 4-MU ester); and optionally (iii) a non-buffering salt; and/or (iv) a non-denaturing detergent not having an ester-bond, wherein the detergent is a non-ionic or zwitter-ionic detergent.

In certain embodiments the hydrophilic substrate HPTS ester in the kit is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof (such as 1-octanoyloxy-pyrene-3,6,8-trisulfonic acid trisodium salt (OPTS), 1-nonaoyloxy-pyrene-3,6,8-trisulfonic acid trisodium salt or 1-decanoyloxy-pyrene-3,6,8-trisulfonic acid trisodium salt); and/or the lipophilic substrate 4-MU ester is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate. The kit according to the invention may further one or more microtiter plate having 96 wells or a multiple of 96 wells.

In yet another aspect the invention relates to a use of a hydrophilic substrate HPTS ester or a hydrophilic substrate HPTS ester and a lipophilic substrate 4-MU ester as a substrate for detecting in an assay carboxylesterase activity or carboxylesterase and lipase activity (respectively) of a contaminating host cell protein in a sample comprising a recombinant protein of interest produced in a eukaryotic cell in cell culture, preferably wherein the recombinant protein is produced in a CHO cell and the at least one contaminating host cell protein is a CHO host cell protein (CHOP), wherein the hydrophilic substrate is a saturated unbranched-chain fatty acid (C6-C12) ester of 1-hydroxypyrene-3,6,8-trisulfonic acid or a salt thereof (substrate HPTS ester), and wherein the lipophilic substrate is a saturated unbranched-chain fatty acid (C6-C16) 4-MU ester.

In yet another aspect the invention relates to a use of a hydrophilic substrate HPTS ester as a substrate for detecting in an assay carboxylesterase activity of at least one contaminating host cell protein in a sample comprising a recombinant protein of interest produced in a eukaryotic cell in cell culture, preferably wherein the recombinant protein is produced in a CHO cell and the at least one contaminating host cell protein is a CHO host cell protein (CHOP), wherein the hydrophilic substrate is a saturated unbranched-chain fatty acid (C6-C12) ester of 1-hydroxypyrene-3,6,8-trisulfonic acid or a salt thereof (substrate HPTS ester).

### DESCRIPTION OF THE FIGURES

FIG. 1: (A) Schematic representation of the family of esterases potentially involved in polysorbate degradation. (B) Schematic representation illustrating the esterases detected by the novel OPTS assay and the 4-MUD assay showing a small overlap and otherwise complementing groups of carboxyl esterases and lipases. (C) Schematic representation of the hydrolysis reaction for the substrate 4-methylumbelliferyl decanoate (4-MUD) forming the fluorophore 4-methylumbelliferone (4-MU). (D) Schematic representation of the hydrolysis reaction for the substrate 1-octanoyloxy-pyrene-3,6,8-trisulfonic acid (OPTS) trisodium salt forming the fluorophore 1-hydroxypyrene-3,6,8-trisulfonic acid (HPTS) trisodium salt.

FIG. 2: (A) Spectra of excitation wavelengths 300-500 nm of HPTS at emission wavelength 520 nm for pH values 4-8 (top), spectrum of emission wavelengths 420-620 nm at excitation wavelength of 415 for pH=5.5 (bottom). (B) Calibration curve for the different pH values (4-8) of the HPTS concentration range of 1.46-3000 nM (top), calibration line for pH=7 of the HPTS concentration range 93.75-6000 nM (bottom).

FIG. 3: Kinetic measurement of a 25 μM OPTS solution with AMT buffer pH=5.5 (filled square) and pH=8 (open triangle) over a period of two hours

FIG. 4: Kinetic measurements of the reaction mixture with 1 mg*mL⁻¹ PPL, 25 μM OPTS and AMT (pH=7) for 30 minutes.

FIG. 5: Kinetic measurements of the reaction mixture with 1 mg*mL⁻¹ PPL, 25 μM OPTS and AMT (pH=7) (A) in the presence or absence of 10 mM CHAPS for 30 minutes and (B) in the presence of the indicated concentrations of Tween 20.

FIG. 6: Kinetic measurements of the reaction mixture with 1 mg*mL⁻¹ PPL, 100 μM OPTS in 150 mM Tris, 0.25% (w/v) Triton X-100, 0125% (w/v) gum Arabic, pH=8 (TTG buffer as described for the esterase activity assay of WO 2020/047416 A2) and AMT buffer at pH=8 for 30 min (A) in the presence or absence of 10 mM CHAPS for 30 minutes.

FIG. 7: Kinetics measurements of 9 different antibody preparations with 25 μM OPTS, AMT buffer (pH=5.5), 75 μL of the respective antibody solution (labeled 1-9) and 300 μL ad. H₂O. The kinetics were recorded at 25° C. for 30 min with n=2 measurements, the results represent the arithmetic mean and the error bar represents the maximum error.

FIG. 8: Kinetics measurements of 9 different antibody preparations with 30 μM 4-MUD substrate, AMT buffer (pH=5.5), 75 μL of the respective antibody solution (labeled 1-9) and 300 μL ad. H₂O. The kinetics were recorded at 25° C. for 30 min with n=2, the results represent the arithmetic mean and the error bar the maximum error.

FIG. 9: Kinetics measurements with 25 μM OPTS (filled square) or 30 μM 4-MUD substrate (open circle), AMT buffer (pH=5.5), 75 μL of the respective antibody solution (labeled 1-9) and 300 μL ad. H₂O. Kinetics were recorded at 25° C. for 30 min with n=2, results represent the arithmetic mean and the error bar represents the Maximum error.

FIG. 10: Kinetics measurements with 30 μM 4-MUD substrate, AMT buffer (pH=5.5), 75 μL of the respective antibody solution (labeled 1-9), 10 μM oristat (squares) or 1 mM PMSF (circles) or 10 mM EDTA (triangles) and 300 μL ad. H₂O. Kinetics were recorded at 25° C. for 30 min with n=2, the results represent the arithmetic mean and the error bar represents the maximum error.

FIG. 11: Kinetics measurements of a lipase with 25 μM OPTS (filled square) or 30 μM 4-MUD substrate (open circles), AMT (pH=5.5), without and with different inhibitors (10 μM oristat, 1 mM PMSF, 10 mM EDTA) and 300 μL ad. H₂O. The final lipase concentration is 0.007 mg*mL⁻¹. The activities correspond to the mean activity units and the error bar corresponds to the maximal error for n=2 measurements.

FIG. 12: pH profile of a lipase measured in a 25 μM OPTS solution (filled squares) and a 30 μM 4-MUD substrate solution (open circles) with AMT at the indicated pH and 0.007 mg*mL⁻¹ lipase. The activities in unit are plotted against the pH of the buffer and the error corresponds to the maximum error at n=2 measurements.

FIG. 13: pH profile of Ab9 preparation measured in a 25 μM OPTS solution (filled squares) and a 30 μM 4-MUD substrate solution (open circles) with AMT at the indicated pH. The activities in Unit are plotted against the pH of the buffer and the error corresponds to the maximum error for n=2 measurements.

FIG. 14: (A) pH profile of 2 preparation measured in a 25 μM OPTS solution (filled squares) and a 30 μM 4-MUD substrate solution (open circles) with AMT at the indicated pH and (B) scaling to the measurement results with 4-MUD substrate. The activities in unit are plotted against the pH of the buffer and the error corresponds to the maximum error for n=2 measurements.

FIG. 15: (A) pH profile of 5 preparation measured in a 25 μM OPTS solution (filled squares) and a 30 μM 4-MUD substrate solution (open circles) with AMT at the indicated pH and (B) scaling to the measurement results with coumarin substrate. The activities in unit are plotted against the pH of the buffer and the error corresponds to the maximum error for n=2 measurements.

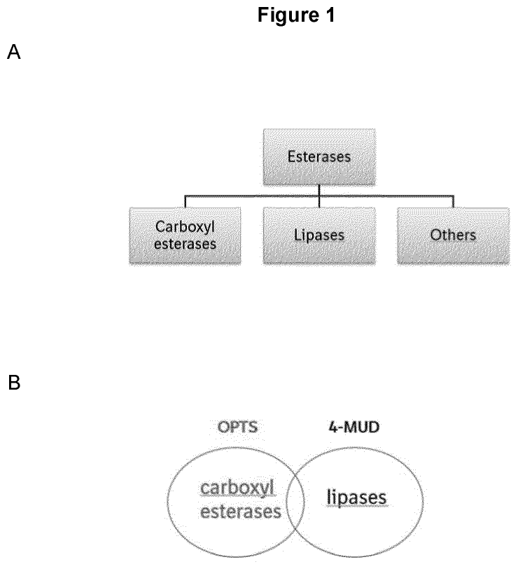

### DETAILED DESCRIPTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. Furthermore, singular and plural forms are not used in a limiting way. As used herein, the singular forms “a”, “an” and “the” designate both the singular and the plural, unless expressly stated to designate the singular only.

The term “sample” as used herein refers to any sample comprising a recombinant protein of interest, wherein the recombinant protein of interest is produced in a eukaryotic cell in cell culture: The at least one sample may, e.g., be a harvested cell culture fluid (HCCF) or a cell lysate, an in-process control (IPC) sample, an ultrafiltration/diafiltration sample (UF/DF sample), a drug substance (also referred to as bulk drug substance) sample or a drug product sample comprising a recombinant protein of interest, such as an antibody, an antibody fragment, an antibody derived molecule or a fusion protein (e.g., an Fc fusion protein). As used herein, the recombinant protein of interest comprised in the sample is not a carboxylase or a lipase and/or does not comprise carboxylase or lipase activity. Thus, any carboxylase or lipase activity detected in the sample is contaminating carboxylase or lipase activity and/or derived from at least one contaminating protein having carboxylase or lipase activity, such as host cell proteins (HCPs) derived from the eukaryotic cell.

The term “contaminating” or “contamination” as used herein refers to the presence of an undesired and/or unintentional substance, such as endogenous proteins produced by the host cell also referred to as host cell proteins, particularly host cell proteins with esterase (carboxylesterase or lipase) activity, which is present only as a trace component in comparison to the recombinant proteins of interest, such as antibodies or antibody-like compounds. In the context of the present invention, a hydrolytic and particularly a lipase or carboxylesterase activity, is undesired due to its polysorbate degrading potential that may be co-purified with the recombinant protein of interest. This applies especially to finally formulated protein preparations which advantageously comprise such unwanted factors only to less than 1% (w/w), preferably less than 0.1% (w/w), more preferably less than 0.01% (w/w) in comparison to total protein content.

The term “lipase activity” as used herein, refers to the activity of a substance, typically a protein (enzyme) that catalyzes the hydrolysis of an ester bond in lipids, such as fatty acid esters. The term “carboxylesterase activity” as used herein refers to the activity of a substance, typically a protein (enzyme) that catalyzes the hydrolysis of an ester bond in a carboxylic ester. A lipase or a carboxylesterase is a hydrolase enzyme that splits esters into an acid and an alcohol in a chemical reaction with water, also referred to as hydrolysis. Many lipases and carboxylesterases belong to the class of carboxylic ester hydrolases (EC 3.1.1). While carboxylesterases form a separate class of carboxylesterase (EC 3.1.1.1), lipases may be, without being limited thereto, a triacylglycerol lipase (EC 3.1.1.3), a phospholipase A2 (EC 3.1.1.4), a lysophospholipase (EC 3.1.1.5), an acylglycerol lipase (EC 3.1.1.23), galactolipase (EC 3.1.1.26), phospholipase A1 (EC 3.1.1.32), lipoprotein lipase (EC 3.1.1.34) or hormone-sensitive lipase (EC 3.1.1.79); a phosphoric diester hydrolase (EC 3.1.4) such as phospholipase D (EC 3.1.4.4), a phosphoinositide phospholipase C (EC 3.1.4.11), glycosylphosphatidylinositol phospholipase D (EC 3.1.4.50) or N-acetylphosphatidylethanolamine-hydrolysing phospholipase D (EC 3.1.4.54); or glycosphingolipid deacylase (EC 3.5.1.69).

A carboxylesterase or a lipase in the context of the present invention is a contaminant that is undesired and often difficult to remove and may or may not mediate polysorbate degrading enzyme activity. The contaminating carboxylesterase may be immunogenic and/or has polysorbate degrading activity, such as sialic acid acetylesterase (SIAE). Carboxylesterases prefer hydrophilic substrates and are therefore detected using the substrate HPTS ester assay. Similarly, the contaminating lipase may be immunogenic and/or has polysorbate degrading activity, such as PLBL2, LPL, LPLA2. PLA2G7, LYPLA2 and LIPA. Lipases prefer lipophilic substrates and are therefore detected using the substrate 4-methylumbelliferyl (4-MU) ester assay. The term “hydrolase activity” as used herein is the more general term, including lipase and carboxylesterase activity, but also refers to hydrolysis of compounds other than lipids or carboxylesters, such as thioester hydrolase (EC 3.1.2), such as palmitoyl protein thioesterase 1 (PPT1), which may also be detected by the combined assay disclosed herein.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties.

The term “recombinant protein” as used herein relates to a protein generated by recombinant techniques, such as molecular cloning and may also be referred to as recombinant protein of interest. As used herein, the recombinant protein is the protein of interest, e.g., in a sample to be purified. Recombinant techniques bring together genetic material from multiple sources or create sequences that do not naturally exist. A recombinant protein is typically based on a sequence from a different cell or organism or a different species from the recipient host cell used for production of the protein in cell culture, e.g., a CHO cell or a HEK 293 cell, or is based on an artificial sequence, such as a fusion protein. In the context of the present invention the recombinant protein is the protein of interest, preferably a therapeutic protein, such as an antibody, an antibody fragment, an antibody derived molecule (e.g., scFv, bi- or multi-specific antibodies) or a fusion protein (e.g., an Fc fusion protein). Thus, in one embodiment the recombinant protein is selected from the group consisting of an antibody, an antibody fragment, an antibody derived molecule and a fusion protein.

The term “eukaryotic cell” as used herein refers to cells that have a nucleus within a nuclear envelop and include animal cells, human cells, plant cells and yeast cells. In the present invention a “eukaryotic cell” particularly encompasses mammalian cell, such as Chinese hamster ovary (CHO) cell or HEK293 cell derived cells, and yeast cells. Mammalian cells as used herein refer to cells, particularly cell lines, of mammalian origin. In the present invention a “mammalian cell” particularly encompasses human or rodent cells, and in most cases refers to a Chinese hamster ovary (CHO) cell or derivatives thereof. Cells as referred to herein are cells maintained in culture and do not relate to primary cells, but cell lines or cell line derived cells, i.e., to immortalized cells.

The term “drug substance (DS)” refers to the formulated active pharmaceutical ingredient (API) with excipients. The API mediates the therapeutic effect in the body as opposed to the excipients, which assist with the delivery of the API. In the case of biologic therapeutics, the formulated API with excipients typically means the API in the final formulation buffer at a concentration of at least the highest concentration used in the final dosage form, also referred to as drug product.

The term “drug product”, abbreviated as DP, as used herein refers to the final marketed dosage form of the drug substance for example a tablet or capsule or in the case of biologics typically the solution for injection in the appropriate containment, such as a vial or syringe. The drug product may also be in a lyophilized form.

The term “polysorbate 20” as used herein refers to a non-ionic polysorbate-type detergent, which is a laureate ester of sorbitol and its anhydrides, copolymerized with approximately 20 moles of ethylene oxide for each mole of sorbitol and sorbitol anhydrides (polyoxyethylene (20) sorbitan monolaurate; CAS number: 9005-64-5). It is also known as Tween 20. Its stability and relative non-toxicity allow it to be used as a surfactant/detergent and emulsifier in a number of domestic, scientific analyses. Polysorbate 20 can be used as washing agent in immunoassays, Western blots and ELISA. It can further be used in pharmacological applications, such as pharmaceutical formulations, particularly for biologics, such as antibodies and Fc-fusion proteins. Particularly it helps to prevent non-specific antibody binding.

The term “polysorbate 80” as used herein refers to a non-ionic polysorbate-type detergent, which is a mixture of partial esters of fatty acids, mainly oleic acid, with sorbitol and its anhydrides ethoxylated with approximately 20 mmoles of ethylene oxide for each mole of sorbitol and sorbitol anhydrides (polyoxyethylene (20) sorbitan monooleate, CAS number: 9005-65-6). It is also known as Tween 80 and has a similar use as polysorbate 20.

The term “therapeutic protein” as used herein refers to proteins that can be used in medical treatment of humans and/or animals. These include, but are not limited to antibodies, growth factors, blood coagulation factors, vaccines, interferons, hormones and fusion proteins.

The term “produced” as used herein relates to the production of the recombinant protein of interest, preferably a therapeutic protein, in a eukaryotic cell, preferably a yeast cell or a mammalian cell, in cell culture. The person skilled in the art knows how to produce recombinant proteins in cells using fermentation. The production of recombinant proteins comprises cultivating the eukaryotic cell expressing the recombinant protein of interest in cell culture. Cultivating the eukaryotic cell expressing the recombinant protein in cell culture comprises maintaining the eukaryotic cells in a suitable medium and under conditions that allow growth and/or protein production/expression. The recombinant protein may be produced by fed-batch or continuous cell culture. Thus, the eukaryotic cells may be cultivated in a fed-batch or continuous cell culture or a combination thereof, preferably in a fed-batch cell culture.

The term “expressing a recombinant protein” as used herein refers to a cell comprising a DNA sequence coding for the recombinant protein of interest, which is transcribed and translated into the protein sequence including post-translational modifications, i.e., resulting in the production of the recombinant protein in cell culture.

The term “about” as used herein refers to a variation of 10% of the value specified, for example, about 50% carries a variation from 45 to 55%.

The term “detecting carboxylesterase activity of the at least one contaminating host cell protein” as used herein refers to a step of measuring hydrolysis of the substrate HPTS ester by detecting the fluorescence intensity of the released chromophore 1-hydroxypyrene-3,6,8-trisulfonic acid or a salt thereof (HPTS). Thus, “detecting carboxylesterase activity of the at least one contaminating host cell protein by measuring hydrolysis of the substrate HPTS ester and detecting the fluorescence intensity of the released chromophore 1-hydroxypyrene-3,6,8-trisulfonic acid or a salt thereof (HPTS)” is used synonymously herein with “detecting carboxylesterase activity of the at least one contaminating host cell protein comprising (or through/via) measuring hydrolysis of the substrate HPTS ester by detecting the fluorescence intensity of the released chromophore 1-hydroxypyrene-3,6,8-trisulfonic acid or a salt thereof (HPTS)”. The step of measuring hydrolysis of the substrate HPTS ester is to be understood to be carried out by detecting the fluorescence intensity of the released chromophore 1-hydroxypyrene-3,6,8-trisulfonic acid or a salt thereof (HPTS).

Similarly, the term “detecting lipase activity of the at least one contaminating host cell protein” as used herein refers to and is carried out by a step of measuring hydrolysis of the substrate 4-MU ester by detecting the fluorescence intensity of the released chromophore 4-MU. Thus, “detecting lipase activity of the at least one contaminating host cell protein by measuring hydrolysis of the substrate 4-MU ester and detecting the fluorescence intensity of the released chromophore 4-MU” is used synonymously herein with “detecting carboxylesterase activity of the at least one contaminating host cell protein comprising (or through/via) measuring hydrolysis of the substrate 4-MU ester by detecting the fluorescence intensity of the released chromophore 4-MU”. The step of measuring hydrolysis of the substrate 4-MU ester is to be understood to be carried out by detecting the fluorescence intensity of the released chromophore 4-MU.

### A Method for Detecting Carboxylesterase Activity of Contaminating Host Cell Protein

The present invention relates to a method (an in vitro method) for detecting carboxylesterase activity of contaminating host cell protein in a sample comprising a recombinant protein of interest produced in a eukaryotic cell comprising (a) providing at least one sample comprising a recombinant protein of interest produced in a eukaryotic cell in cell culture and at least one contaminating host cell protein; (b) contacting the at least one sample with a reaction solution (comprising a hydrophilic substrate) to form a reaction mixture, wherein the reaction solution comprises: (i) a buffer having a pH of about pH 4 to about pH 8, (ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and (iii) optionally a non-buffering salt; (c) incubating the sample and the substrate in the reaction mixture; (d) detecting carboxylesterase activity of the at least one contaminating host cell protein by measuring hydrolysis of the substrate HPTS ester and detecting the fluorescence intensity of the released chromophore 1-hydroxypyrene-3,6,8-trisulfonic acid or a salt thereof (HPTS); optionally measuring hydrolysis by detecting the fluorescence intensity of the released chromophore HPTS over time, while incubating the sample and the substrate in the reaction mixture according to step (c). Thus, detecting carboxylesterase activity of the at least one contaminating host cell protein in step (d) comprises measuring hydrolysis of the substrate HPTS ester by detecting the fluorescence intensity of the released chromophore 1-hydroxypyrene-3,6,8-trisulfonic acid or a salt thereof (HPTS), optionally measuring hydrolysis by detecting the fluorescence intensity of the released chromophore HPTS over time, while incubating the sample and the substrate in the reaction mixture according to step (c). The reaction solution used in the method of the invention is an aqueous reaction solution. The person skilled in the art would understand that in step (b) a portion of the at least one sample is used for each reaction mixture. Further, more than one sample may be analysed in parallel and/or more than one portions of the at least one sample may be analysed in parallel. These more than one sample and/or more than one portions may be analysed under the same conditions or under separate conditions, such as analysing a portion (or duplicates or triplicates thereof) of the at least one sample each at different pH values. This method is particularly suitable for small volumes and high throughput analysis. Thus, multiple reaction mixtures may be analysed in parallel, such as in microtiter plate having 96 wells or a multiple of 96 wells. Preferably each reaction mixture volume is 300 μl or less. Preferably each reaction mixture volume is 300 μl or less.

The assay read out may be as fast as 20 min or even faster. Thus, in certain embodiments the sample and the substrate in the reaction mixture are incubated for less than 5 hours, less than 3 hours, less than 2 hours, or less than 0.5 hours. For obtaining enough data points it is advisable to incubate the sample and the substrate in the reaction mixture for at least 1 min, at least 2 min or at least 5 min. Thus, the sample and the substrate in the reaction mixture may be incubated for any time period between 2 min and 5 hours, 2 min and 3 hours, 2 min and 2 hours, or 2 min and 0.5 hours or between 2 min and less than 5 hours, less than 3 hours, less than 2 hours, or less than 0.5 hours. In certain embodiments multiple reaction mixtures are analysed in parallel, such as 12 or more, 24 or more, 36 or more, 72 or more or 96 or more, and/or the reaction mixture has a volume of 300 μl or less. The at least one sample may be a HCCF, an in-process control (IPC) sample, a UF/DF sample, a drug substance or a drug product. According to the invention the recombinant protein of interest in the sample is not a carboxylesterase and/or does not comprise carboxylesterase activity.

In certain embodiments, the fluorescence of the released chromophore HPTS is determined using an excitation wavelength within a range of 401-405 nm and an emission wavelength within a range of 510-516 nm, preferably an excitation wavelength within a range of 402-404 nm and an emission wavelength within a range of 510-514 nm, more preferably an excitation wavelength of 403 nm and an emission wavelength of 512 nm.

Hydrolysis may be stopped at certain time points prior to detection of the fluorescence intensity of the released chromophore HPTS. Alternatively and preferably, the fluorescence intensity of the released chromophore HPTS may be detected in real-time without stopping hydrolysis of the hydrophilic substrate HPTS ester. In certain embodiments, the fluorescence intensity of the released chromophore HPTS is detected without stopping hydrolysis of the hydrophilic substrate HPTS ester. In certain embodiments hydrolysis is measured by detecting the fluorescence intensity of the released chromophore HPTS over time, while incubating the sample and the substrate in the reaction mixture according to step (c).

The hydrophilic substrate comprising the chromophore HPTS is in the form of saturated unbranched-chain fatty acid (C6 to C12) HPTS ester, wherein the acyl chain of the saturated unbranched-chain fatty acid has from C6 to C12 carbon atoms. The abbreviation “HTPS” as used herein refers to 1-hydroxypyrene-3,6,8-trisulfonic acid or a salt thereof, preferably a trisodium salt. Similarly, the abbreviation “substrate HPTS ester” as used herein refers to a saturated unbranched-chain fatty acid (C6-C12) ester of 1-hydroxypyrene-3,6,8-trisulfonic acid or a salt thereof, preferably a trisodium salt. The abbreviation “OPTS” as used herein refers to 1-octanoyloxy-pyrene-3,6,8-trisulfonic acid or a salt thereof, preferably a trisodium salt. The saturated unbranched-chain fatty acid (C6 to C12) HPTS ester, e.g., 1-octanoyloxy-pyrene-3,6,8-trisulfonic acid (OPTS) trisodium salt is hydrolysed in the presence of a carboxylesterase to the saturated unbranched-chain fatty acid (C6 to C12), such as octanoic acid and -hydroxypyrene-3,6,8-trisulfonic acid (HPTS) trisodium salt as shown below:

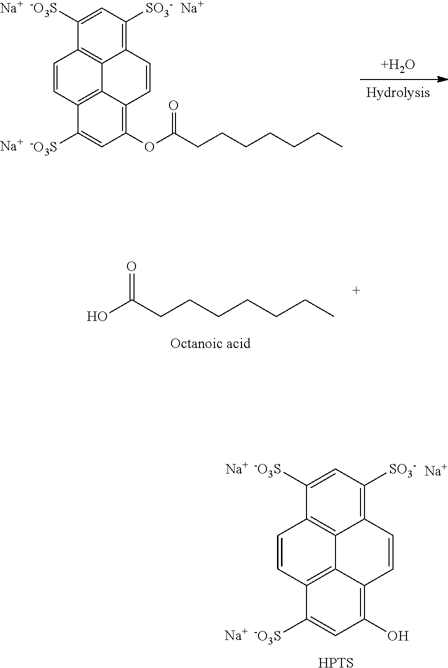

This substrate mimics key feature of polysorbate, i.e., a fatty acid ester bond and a long acyl chain. Polysorbate 20 is an ester of the fatty acid lauric acid, a saturated unbranched-chain fatty acid. Polysorbate 80 in comparison is an ester of the fatty acid oleic acid, an unsaturated fatty acid. The hydrophilic substrate is a saturated unbranched-chain fatty acid (C6-C12) ester of 1-hydroxypyrene-3,6,8-trisulfonic acid or a salt thereof (substrate HPTS ester). Autohydrolysis increases for substrate HPTS ester with shorter saturated unbranched chain fatty acid (<C6), while substrate HPTS ester with longer saturated unbranched chain fatty acid (>C12) become more lipophilic and are hence less favorable substrates for carboxylesterases. Thus, the substrate HPTS ester is selected from the group consisting of 1-hexanoyloxy-pyrene-3,6,8-trisulfonic acid, 1-heptanoyloxy-pyrene-3,6,8-trisulfonic acid, 1-octanoyloxy-pyrene-3,6,8-trisulfonic acid (OPTS), 1-nonaoyloxy-pyrene-3,6,8-trisulfonic acid, 1-decanoyloxy-pyrene-3,6,8-trisulfonic acid, 1-undecanoyloxy-pyrene-3,6,8-trisulfonic acid, 1-dodecanoyloxy-pyrene-3,6,8-trisulfonic acid and salts thereof. Preferably, the substrate HPTS ester is a C8-C10 ester, such as selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof. The salt may be any salt, such as a sodium or a potassium salt. In a preferred embodiment the chromophore 1-hydroxypyrene-3,6,8-trisulfonic acid or a salt thereof is 1-hydroxypyrene-3,6,8-trisulfonic acid trisodium salt and the substrate HPTS ester is selected from the group consisting of 1-hexanoyloxy-pyrene-3,6,8-trisulfonic acid trisodium salt, 1-heptanoyloxy-pyrene-3,6,8-trisulfonic acid trisodium salt, 1-octanoyloxy-pyrene-3,6,8-trisulfonic acid trisodium salt (OPTS), 1-nonaoyloxy-pyrene-3,6,8-trisulfonic acid trisodium salt, 1-decanoyloxy-pyrene-3,6,8-trisulfonic acid trisodium salt, 1-undecanoyloxy-pyrene-3,6,8-trisulfonic acid trisodium salt and 1-dodecanoyloxy-pyrene-3,6,8-trisulfonic acid trisodium salt, more preferably of the group consisting of 1-octanoyloxy-pyrene-3,6,8-trisulfonic acid (OPTS) trisodium salt, 1-nonaoyloxy-pyrene-3,6,8-trisulfonic acid trisodium salt and 1-decanoyloxy-pyrene-3,6,8-trisulfonic acid trisodium salt.

The substrate HPTS ester is a hydrophilic substrate and is therefore freely soluble in water. Organic solvents and detergents tend to interfere with the hydrophilic substrate HPTS ester assay and/or quenches the chromophore HPTS. The hydrophilic substrate HPTS ester is therefore preferably dissolved in an aqueous solution, such as water or an aqueous buffer, more preferably in an aqueous solution not comprising DMSO or DMF.

Suitable hydrophilic substrate concentration (in the reaction mixture) in the methods of the present invention may be about 1 μM to about 1 mM. Preferably the hydrophilic substrate HPTS ester is used at a final concentration in the reaction mixture of 50 μM, more preferably of 30 μM or less. solution is Thus, in certain embodiments the substrate is provided at a final concentration in the reaction mixture of about 1 μM to about 1 mM, preferably about 1 μM to about 300 μM, preferably about 1 μM to about 50 μM, preferably about 1 μM to about 30 μM, more preferably about 3 μM to about 30 μM. In certain embodiments the substrate is provided as stock solution in an aqueous solution. The term “aqueous solution” as used herein means in a water-based solution, such as water or a buffer (such as the buffer used in the reaction solution), preferably not comprising an organic solvent. The stock solution is added at about 1% to about 10% (v/v) of the reaction mix.

Moreover, it is essential that the reaction solution comprising the substrate HPTS ester does not comprise a detergent above its critical micelle concentration (CMC), preferably the reaction solution comprising the substrate HPTS ester does not comprise a detergent (e.g., non-ionic detergent, non-ionic detergent or a zwitterionic detergent). This particularly includes the non-denaturing detergent (non-ionic or zwitter-ionic detergent) not having an ester bond as described herein, as well an ethoxylate and/or detergents comprising a polyethylene glycol group (such as glycol tert-octylphenyl ether (Triton X-100, CAS No. 9002-93-1) and/or polyethylene glycol nonylphenyl ether (NP-40, CAS No. 9016-45-9)) and/or an aromatic ring. Thus, in a specific embodiment the reaction solution does not comprise a detergent, such as Triton X-100, NP-40 or CHAPS, particularly not above the respective CMC. Preferably no detergent at or above its critical micelle concentration (CMC) is present in the reaction solution or in the reaction solution comprising the hydrophilic substrate and the reaction mixture, more preferably no detergent is present in the reaction solution or in the reaction solution and the reaction mixture. In certain preferred embodiments, the reaction solution as used in the method according to the invention, comprising (i) the buffer, (ii) the hydrophilic substrate, and optionally (iii) a non-buffering salt, does not contain a detergent. The person skilled in the art will understand that since the sample to be analysed may contain a detergent, the reaction mixture may contain small amounts of detergent following contacting of the reaction solution the sample. The hydrophilic substrate HPTS ester assay is intended for in-process control samples of recombinant protein of interest manufacture as a fast high-throughput assay for purification train development and optimization. Most in-process control samples do not contain detergents, such as polysorbate 20 or polysorbate 80. The samples that may contain detergents are mainly the final drug substance and possibly the harvested cell culture fluid (HCCF) due to antifoaming agents. However, considering the high hydrolase activity in HCCF (prior to purification), samples would need to be diluted and hence potential antifoaming agents reach concentrations that would not interfere with the assay. Thus, the final reaction mixture (sample and reaction solution comprising the hydrophilic substrate) may comprise low concentrations of polysorbate 20 or 80 derived from the sample as described herein. With regard to the final product, most drug substance samples have an uncritical polysorbate concentration following dilution in the reaction mix (such as about 50 μg/ml polysorbate 20 or less). Alternatively, if required, UF/DF samples, i.e., prior to addition of polysorbate, may be used for determining hydrolytic activity. The polysorbate 20 or polysorbate 80 concentration in the reaction mixture should preferably be below CMC, and/or not exceed about 100 μg/ml, preferably about 75 μg/ml, more preferably about 50 μg/ml for polysorbate 20 (or not exceed about 60 μg/ml, preferably about 50 μg/ml, preferably about 30 μg/ml, more preferably about 20 μg/ml for polysorbate 80). However, we also note in this context that the assay is particularly useful for high throughput analysis of IPC samples in small volumes for process optimization, which typically do not contain polysorbate.

It was further observed that histidine may inhibit the method according to the invention using the hydrophilic substrate HPTS ester. Histidine is often used in formulation buffers. In case histidine is present in the sample, it should be dialysed prior to the method according to the invention to remove histidine from the sample. Methods for preparative dialysis are well known in the art. For example, an antibody solution may be dialysed against the 500-fold volume of 0.002% (w/v) NaCl solution with gentle stirring using a pre-wettened dialysis cassette and replacing the 0.002% (w/v) NaCl solution at least once. Each dialysis step is maintained for about 1-2 hours. A histidine concentration of less than 200 μM was found to be acceptable. Thus, in one embodiment a sample comprising the protein of interest is dialysed until a concentration of less than 200 μM histidine is reached. Alternative methods would be the use of a dextran-epichlorohydrin copolymer (Sephadex®) column (size exclusion chromatography) or affinity chromatography. In certain embodiments the sample comprising a recombinant protein of interest produced in a eukaryotic cell in cell culture and at least one contaminating host cell protein comprises less than 200 μM histidine, preferably less than 100 μM histidine. Thus, with regard to the final product, if required, the drug substance sample may be either dialysed, or UF/DF samples, i.e., prior to addition of histidine (and polysorbate), may be used for determining hydrolytic activity. However, we note in this context that the assay is particularly useful for high throughput analysis of IPC samples in small volumes for process optimization, which typically do not contain histidine.

The method according to the invention may further comprise analysing the at least one sample using a lipophilic substrate 4-MU ester. Thus, this method may further comprise detecting lipase activity in the sample comprising a recombinant protein of interest produced in a eukaryotic cell and at least one contaminating host cell protein. In certain embodiments, the method according to the invention further comprises (bi) contacting the at least one sample comprising a recombinant protein of interest produced in a eukaryotic cell in cell culture and at least one contaminating host cell protein of step (a) in a separate reaction set-up with a reaction solution comprising a lipophilic substrate to form a reaction mixture, wherein the reaction solution comprises: (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester (substrate 4-MU ester), (iii) optionally a non-buffering salt, and (iv) a non-denaturing detergent not having an ester-bond, wherein the detergent is a non-ionic or zwitterionic detergent; (ci) incubating the sample and the substrate in the reaction mixture of step (bi); (di) detecting lipase activity of the at least one contaminating host cell protein by measuring hydrolysis of the substrate 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (ci). Thus, detecting lipase activity of the at least one contaminating host cell protein in step (di) comprises measuring hydrolysis of the substrate 4-MU ester by detecting the fluorescence intensity of the released chromophore 4-MU, optionally measuring hydrolysis by detecting the fluorescence intensity of the released chromophore 4-MU overtime, while incubating the sample and the substrate in the reaction mixture according to step (ci). The person skilled in the art would understand that in step (bi) a portion of the at least one sample is used for each reaction mixture. More specifically in step (b) a portion of the at least one sample is used for each reaction mixture and in step (bi) another portion of the at least one sample is used for each reaction mixture. Further, more than one sample may be analysed in parallel and/or more than one portions (or fractions) of the at least one sample may be analysed in parallel using the substrate HPTS ester and optionally the substrate 4-MU ester. These more than one sample and/or more than one portions may be analysed under the same conditions or under separate conditions, such as analysing a portion (or duplicates or triplicates thereof) of the at least one sample each at different pH values. This method is particularly suitable for small volumes and high throughput analysis. Thus, multiple reaction mixtures may be analysed in parallel, such as in microtiter plate having 96 wells or a multiple of 96 wells. Preferably each reaction mixture volume is 300 μl or less.

Analysing the at least one sample using the hydrophilic substrate HPTS ester and addition the lipophilic substrate 4-MU ester broadens the spectrum of enzymes capture by the assay that are potentially involved in polysorbate degradation. The hydrophilic substrate and the hydrophilic substrate complement each other by detecting enzymes with hydrolytic activity and a rather lipophilic substrate specificity (such as lipases) and enzymes with hydrolytic activity and a rather hydrophilic substrate specificity (such as carboxylesterases). The substrates are intended to be used in parallel or subsequently using the same at least one sample. The person skilled in the art will understand that a first portion of the at least one sample is contacted with the reaction solution as defined in step (b) and a second portion of the at least one sample is contacted with the reaction solution as defined in step (bi).

The term “different reaction set-up” as used herein refers to a separate reaction, which is different to the reaction using reaction solution comprising the hydrophilic substrate HPTS ester in that it uses a reaction solution comprising a lipophilic substrate 4-MU ester and which is performed in a different vessel or well (i.e., physically separate). The different reaction set-up is typically performed using the same at least one sample or rather each using a portion of the same at least one sample for contacting and analysis. The buffer (and optionally the non-buffering salt) used in the reaction solution comprising the hydrophilic substrate and in the reaction solution comprising the lipophilic substrate is preferably the same. Thus, the reaction solution comprising the hydrophilic substrate and the reaction solution comprising the lipophilic substrate are the same, except for the substrate and the detergent present only in the reaction solution comprising the reaction solution comprising the lipophilic substrate.

The substrate comprising the chromophore 4-MU is in the form of saturated unbranched-chain fatty acid (C6 to C16) 4-MU ester, wherein the acyl chain of the saturated unbranched-chain fatty acid has from C6 to C16 carbon atoms. This substrate mimics key feature of polysorbate, i.e., a fatty acid ester bond and a long acyl chain. Polysorbate 20 is an ester of the fatty acid lauric acid, a saturated unbranched-chain fatty acid. Polysorbate 80 in comparison is an ester of the fatty acid oleic acid, an unsaturated fatty acid. The fluorescence of the released chromophore 4-MU is suitable for detecting the fluorescence in a fluorescence spectrometer or a microplate spectrophotometer. In certain embodiments the fluorescence of the released chromophore 4-MU is determined using an excitation wavelength within a range of 330-340 nm and an emission wavelength of 450 nm.

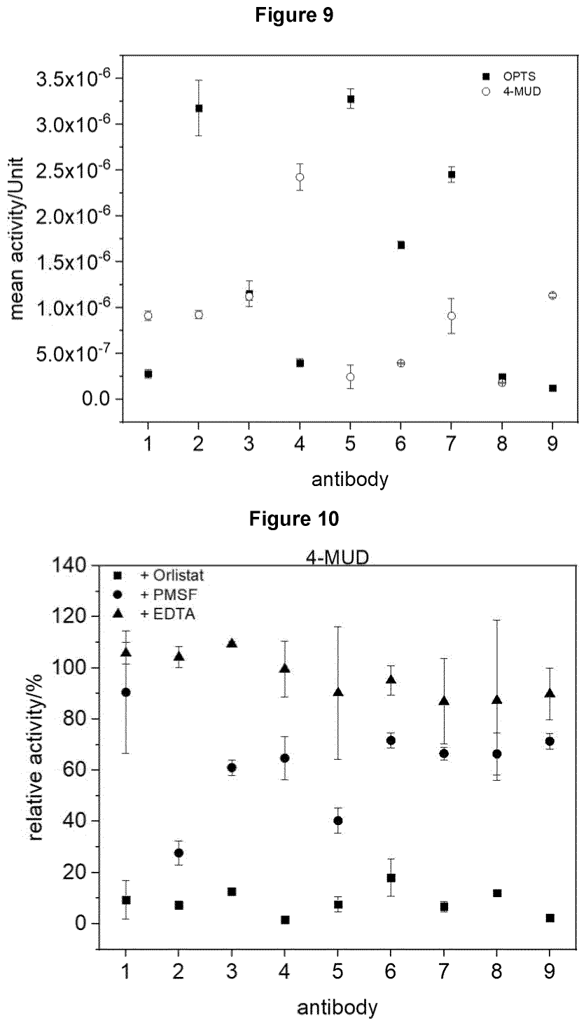

Unsaturated fatty acids are more bulky than saturated fatty acids due to the double bond(s) and further branched-chain fatty acids are more bulky compared to unbranched-chain fatty acids. Lipase activity and/or carboxylesterase activity in a sample comprising a recombinant protein or interest may be mediated by one or more lipases and/or carboxylases and differ between various recombinant proteins of interest, such as individual antibodies (see FIG. 9). Thus, in most cases the contaminating host cell protein(s) with lipase activity and/or carboxylase activity is/are unknown and may be a mixture of more than one protein. Particularly, many lipases, such as triacylglycerol lipases, can be in an open state or in a closed state whereas the active site is shielded from the solvent by a part of the polypeptide chain, the flap or lid. Thus, the active site of many lipases resembles a cavity or the inside of a barrel, which most likely determines substrate specificity. An ester of a saturated unbranched-chain fatty acid (less bulky fatty acid) that is of medium length (C6-C16) of the lipophilic substrate 4-MU ester used in the methods of the invention is therefore likely to capture a broader enzyme spectrum compared to, e.g., oleate having a longer and unsaturated acyl chain. Similarly, an ester of a saturated unbranched-chain fatty acid (less bulky fatty acid) that is of medium length (C6-C12) of the hydrophilic substrate HPTS ester used in the methods of the invention is likely to capture a broader enzyme spectrum compared to fatty acids having a longer and/or unsaturated acyl chain. Preferable the substrate HTPS ester and/or 4-MU ester alone or in combination capture(s) an equal or broader enzyme spectrum compared to PS20 or PS80.

Further, fatty acid esters with shorter acyl chains offer better solubility in water-based (aqueous) reaction mixtures compared to longer chain length fatty acid esters. Consequently, more substrate can be used in the assay mix. More specifically, it was found that solubility of the substrate 4-MU ester becomes strongly limiting at a chain length of C16 or longer. While the substrate HPTS ester is generally more water soluble, without being bound by theory, increasing a chain length to more than C12 renders the substrate more lipophilic, which may shift the enzyme spectrum to more enzymes with more lipophilic substrate specificity, such as lipases, thereby reducing the breadth of the combined enzyme spectrum of the substrate HPTS ester and the substrate 4-MU ester.

Additionally, it was found for the substrate 4-MU ester that the decanoate ester (4-MUD) offers a better resistance to auto-hydrolysis compared to e.g. a butyrate ester (4-MUB). It was found that a chain length up to C5 strongly increased auto-hydrolysis. The C10 fatty acid in 4-MUD was reported to be optimal for use in the assay (WO 2022/049294 A1), but slightly longer or shorter saturated unbranched fatty acid esters, such as saturated unbranched-chain fatty acid (C6 to C16) 4-MU ester or more preferably saturated unbranched-chain fatty acid (C8 to C12) 4-MU ester may similarly be used in the method according to the invention. Similar observations with regard to auto-hydrolysis were made for the hydrophilic substrate HPTS esters. Thus, the hydrophilic substrate HPTS ester used in the method according to the invention has an acyl chain of the saturated unbranched-chain fatty acid from C6 to C12 carbon atoms.

Where additionally a lipophilic 4-MU ester is used in the method according to the invention, it has an acyl chain of the saturated unbranched-chain fatty acid from C6 to C16 carbon atoms. More preferably, the fatty acid is a medium-chain fatty acid and the 4-MU ester is a saturated unbranched-chain fatty acid (C8 to C12) 4-MU ester. In certain embodiments, the lipophilic substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate. In certain preferred embodiments the lipophilic substrate is selected from the group consisting of 4-methylumbelliferyl octanoate, 4-methylumbelliferyl decanoate (4-MUD) and 4-methylumbelliferyl dodecanoate. In a more preferred embodiment, the substrate is 4-MUD. The lipophilic substrate 4-MU ester is typically dissolved as a stock solution (such as a 100× stock solution relative to the concentration in the reaction mixture) in an organic solvent, such as dimethyl sulfoxide (DMSO) or dimethyl formamide (DMF), preferably DMSO. In certain embodiments the lipophilic substrate is provided a stock solution dissolved in an organic solvent selected from DMSO or DMF, preferably DMF.

Suitable lipophilic substrate concentration (in the reaction mixture) in the present invention may be about 1 μM to about 1 mM. Thus, in certain embodiments the substrate is provided at a final concentration in the reaction mixture of about 1 μM to about 1 mM, preferably about 1 μM to about 300 μM, preferably about 1 μM to about 30 μM, more preferably about 3 μM to about 30 μM. In certain embodiments the substrate is provided as a stock solution in an organic solvent, wherein the stock solution is added at about 1% to about 5% (v/v) of the reaction mix.

The method according to certain embodiments comprises contacting the at least one sample with a reaction solution comprising a non-denaturing detergent not having an ester-bond, wherein the detergent is non-ionic or zwitter-ionic surfactant (also referred to herein as “non-denaturing non-ionic or zwitter-ionic detergent not having an ester-bond”). The term “detergent” as used herein refers to a surface-active compound that is able to form micelles. A detergent may also be referred to as a surfactant herein. Detergents are amphiphilic, i.e., comprising both hydrophobic groups (tail) and hydrophilic groups (head). Detergents are typically organic compounds. In aqueous phase, detergents form aggregates, such as micelles, where the hydrophobic tail forms the core of the aggregate and the hydrophilic heads are in contact with the surrounding aqueous liquid. The hydrophobic tail (also referred to as hydrophobic hydrocarbon moiety) therefore has a certain length to form micelles. Thus, detergents as used herein do not encompass organic solvents, such as ethanol or dimethylsulfoxide (DMSO). The tail of most detergents typically consists of one or more hydrocarbon chain, which can be branched, linear or aromatic. The detergent may comprise one or more hydrophobic tail, preferably the detergent comprises one hydrophobic chain (single-tailed detergent). Detergents are commonly classified according to the hydrophilic head group. A non-ionic detergent has no charged groups in their head, an ionic detergent carries a net positive (cationic), or negative (anionic) charge, and a zwitterionic detergent contains two oppositely charged groups. Thus, non-ionic or zwitterionic detergents do not carry a net charge at the hydrophilic head group and are therefore milder in nature. Moreover, in many detergents the hydrophobic tail is inked to the hydrophilic head via an ester bond, as in PS20 or PS80. Moreover, the non-ionic or zwitterionic detergent is a non-denaturing detergent. The term “non-denaturing detergent” as used herein refers to the effect of the detergent with respect to protein structure. A non-denaturing detergent does not disrupt protein-protein interactions, particularly of water-soluble proteins.

Detergents comprising an ester bond are potential substrates to lipases or carboxylesterases and may therefore interfere with the assay. Moreover, denaturation of the proteins with lipase activity and hence interference with the lipase activity in the sample is to be avoided. The detergent to be used in the method according to the invention is therefore a non-denaturing detergent not having an ester-bond, wherein the detergent is a non-ionic or zwitter ionic detergent. Examples for suitable non-denaturing zwitter-ionic detergents are without being limited thereto 3-[(3-cholamidopropyl)dimethylammonio]-1-propanesulfonate (CHAPS), 3-([3-Cholamidopropyl)dimethylammonio]-2-hydroxy-1-propanesulfonate (CHAPSO), CHAPS analogs (such as Big CHAP N,N-bis-(3-D-gluconamidopropyl)deoxycholamide), Zwittergent (different lengths, such as n-Dodecyl-N,N-dimethyl-3-ammonio-1-propanesulfonate (Zwittergent 3-12)) and 3-[N,N-Dimethyl(3-palmitoylaminopropyl)ammonio]-propanesulfonate or other amidosulfobetaine detergents. Examples for suitable non-denaturing, non-ionic detergents are without being limited thereto pyranoside detergents (such as Octyl β-D-glucopyranoside (OGP), Nonyl β-D-glucopyranoside, Dodecyl β-D-maltopyranoside (DDM) or Octyl β-D-thioglucopyranoside), polyoxyethylene (23) lauryl ether (Brij 35) or other Polyoxyethylene ether; saponins (e.g. Digitonin), octylphenoxy polyethoxyethanol (IGEPAL CA-630), poloxamer 188, 338, 407 or tergitol. In certain embodiments, the non-denaturing detergent (non-ionic or zwitter-ionic detergent) not having an ester-bond is not an ethoxylate and/or does not comprise a polyethylene glycol group and/or does not comprise an aromatic ring. In certain embodiments, the non-denaturing non-ionic or zwitter-ionic detergent not having an ester-bond is not an octoxinol-9, specifically not polyethylene glycol tert-octylphenyl ether (Triton X-100, CAS No. 9002-93-1) and/or polyethylene glycol nonylphenyl ether (NP-40, CAS No. 9016-45-9). In preferred embodiments, the detergent is a non-denaturing detergent not having an ester-bond, wherein the detergent is a non-ionic or zwitter-ionic detergent, more preferably the detergent is a non-denaturing detergent (non-ionic or zwitter-ionic detergent) selected from the group consisting of CHAPS (CAS No. 75621-03-3), CHAPSO (CAS No. 82473-24-3), Zwittergent (such as Zwittergent 3-12; CAS No. 14933-08-5) and a saponin (CAS No. 8047-15-2), preferably CHAPS. None of these exemplary suitable detergents exhibit an ester bond or an acyl chain and are therefore not a substrate for lipases. These detergents do not compete with the substrate and hence does not affect sensitivity of the assay. Additionally, the presence of a detergent mediates solubility of the substrate at the used concentrations in water. The person skilled in the art would know how to identify further suitable non-denaturing non-ionic or zwitter-ionic detergent not having an ester-bond by determining its effect on 4-MU ester hydrolysis under assay conditions.

It has been shown that the presence of a detergent (such as 10 mM CHAPS) increases lipase activity and hence improves sensitivity of the assay. Without being bound by theory it is hypothesized that a detergent creates an environment that promotes lipase activity by allowing the rearrangement and opening of the id or flap, which has been described to cover the active site (Grochulski P, Li Y, Schrag JD, et al. Protein Sci 1994; 3:82-91 and Grochulski P, Bouthillier F, Kazlauskas RJ, et al. Biochemistry 1994; 33:3494-500). To achieve this effect, the detergent should be above its critical micelle concentration (CMC).

Thus, according to the invention the non-denaturing detergent (non-ionic or zwitter-ionic) has a final concentration in the reaction mixture above its critical micelle concentration (CMC) in the reaction mixture. CMC represents an important physicochemical characteristic of a given detergent in aqueous solution. Micelles are spherical aggregates whose hydrocarbon groups are to a large extent out of contact with water. The term “critical micelle concentration” or “CMC” as used herein refers to the concentration of a detergent above which micelles are formed (i.e., the maximum monomer concentration) and may be determined according to methods known in the art. For example, a suitable method for determining the CMC is the fluorescence micelle assay (FMA), which uses the partitioning of the fluorescent hydrophobic dye N-phenyl-1-napthylamine (NPN) into detergent micelles. NPN exhibits a low-fluorescence quantum yield in aqueous environments, which increase in more hydrophobic environments such as the core of the micelles. This assay has originally been developed for CMC determination and is also been used to determine the content of polysorbate in biopharmaceuticals as in the examples. An alternative method utilizing enhancement of 1,6-diphenyl-1,3,5-hexatriene (DPH) fluorescence upon micellization is described by Chattopadhyay and Harikumar (FEBS Letters 391 (1996) 199-202).

The CMC for a detergent is derivable from literature and is e.g., about 6 mM for CHAPS, about 8 mM for CHAPSO and about 2-4 mM for Zwittergent 3-12. In certain embodiments, the non-denaturing zwitter-ionic detergent is CHAPS and is provided at a final concentration in the reaction mixture of about 8 mM to about 20 mM, preferably at about 8 mM to about 15 mM, more preferably at about 10 mM. In other embodiments the non-denaturing zwitter-ionic detergent is CHAPSO and is provided at a final concentration in the reaction mixture of about 10 mM to about 20 mM, preferably at about 10 mM to about 15 mM. In yet another embodiment the non-denaturing zwitter-ionic detergent is Zwittergent 3-12 and is provided at a final concentration in the reaction mixture of about 4 mM to about 10 mM, preferably at about 6 mM to about 8 mM. In yet another embodiment the non-denaturing non-ionic detergent is a saponin and is provided at a final concentration in the reaction mixture of about 0.001% to 0.01% (w/v).

Hydrolysis may be stopped at certain time points prior to detection of the fluorescence intensity of the released chromophore 4-MU. Alternatively and preferably, the fluorescence intensity of the released chromophore 4-MU may be detected in real-time without stopping hydrolysis of the lipophilic substrate 4-MU ester. In certain embodiments, the fluorescence intensity of the released chromophore 4-MU is detected without stopping hydrolysis of the lipophilic substrate 4-MU ester. In certain embodiments hydrolysis is measured by detecting the fluorescence intensity of the released chromophore 4-MU over time, while incubating the sample and the substrate in the reaction mixture according to step (ci).

Real-time detection allows measuring hydrolysis over time and hence the specific reaction rate may be determined. In the method according to the invention hydrolysis of substrate HPTS ester or 4-MU ester in the reaction mixture typically follows a pseudo-zero order reaction rate. Detecting fluorescence in real-time therefore allows measurement in a time-frame with a pseudo-zero order reaction rate. Thus, in certain embodiments, the fluorescence intensity of the released chromophore HPTS or 4-MU is detected over time and follows a pseudo-zero order reaction rate. Optionally a reaction mixture that does not meet the requirement of a pseudo-zero order reaction rate is excluded from analysis. A pseudo-zero order reaction rate can be assessed by linear regression analysis. Preferably, samples are run at least in triplicates and individual reaction mixtures are excluded from analysis in case they do not meet a pseudo-zero order reaction rate, e.g. due to bubbles in the well etc., to eliminate outliers. Eliminating outliers as described strongly increases sensitivity of the assay. Calibration curves using defined concentrations of HPTS or 4-MU can be used to calculate the rate of hydrolysis (e.g. nmol/s). Calibration curves with known HPTS or 4-MU concentrations further allow the determination and comparison of reaction velocities at different pH values.

The term “reaction rate” as used herein refers to the velocity of an enzyme converting a substrate into at least one product within a specific period. In some reactions, the rate is apparently independent of the reactant concentration. This means that the rate of the equation is equal to the rate constant, k, of the reaction and is referred to as zero-order reaction. A zero-order kinetics is always an artefact of the conditions under which the reaction is carried out. For this reason, reactions that follow zero-order kinetics are often referred to as pseudo-zero-order reactions.

The method according to the invention may further comprise a step of determining the rate of hydrolysis by detecting the fluorescence intensity of the released chromophore HPTS or 4-MU as relative fluorescent units (RFU) and determining the amount of the released chromophore HPTS or 4-MU (mol/s) by comparing it to a calibration curve generated by using defined concentrations of HPTS or 4-MU. Typically, activity is measured by release of HPTS or 4-MU in nmol/min. Alternatively or in addition a relative value may be calculated compared to an internal standard, such as another sample or preferably a commercially available lipase such as porcine pancreatic lipase (PPL) (or a commercially available crude extract comprising PPL) that serves as a positive control.

Incubation of the sample and the substrate in the reaction mixture allows the potentially present at least one contaminating host cell protein having carboxylesterase activity to hydrolyse the substrate HPTS ester and in a separate sample the potentially present at least one contaminating host cell protein having lipase activity to hydrolyze the substrate 4-MU ester. Incubation is typically from a few minutes to a few hours. In one embodiment hydrolysis is measured by detecting the fluorescence intensity of the released chromophore HPTS or 4-MU over time, while incubating the sample and the substrate in the reaction mixture according to step (c) or (ci), respectively, i.e., in real-time during incubation. Due to the sensitivity of the assay, detection typically starts immediately following step (b) or (bi), respectively. Incubation and hence detection time may depend on the lipase activity present in the sample and does typically not exceed 5 hours, preferably not 3 hours. In certain embodiments, the sample and the substrate in the reaction mixture are incubated for less than 5 hours, less than 3 hours, less than 2 hours, less than 1 hour, or less than 0.5 hours. For obtaining enough data points it is advisable to incubate the sample and the substrate in the reaction mixture for at least about 1 min, at least about 2 min or at least about 5 min. Thus, the sample and the substrate in the reaction mixture may be incubated for any time period between 2 min and 5 hours, 2 min and 3 hours, 2 min and 2 hours, or 2 min and 0.5 hours or between about 2 min and less than 5 hours, less than 3 hours, less than 2 hours, or less than 0.5 hours. Preferably, the sample and the substrate in the reaction mixture are incubated between 20 minutes and 2 hours at a temperature of about 25° C. Since reaction temperature influences reaction time, the reaction temperature should be kept constant during measurement, such as at a constant temperature between 20-37° C., preferably between 22-28° C., more preferably between 24-26° C. In one embodiment, the sample and the substrate in the reaction mixture are incubated for less than 5 hours, less than 3 hours, less than 2 hours or less than 1 hour at a constant temperature between 20-37° C., preferably between 22-28° C., more preferably between 24-26° C. or for any time period between 2 min and 5 hours, 2 min and 3 hours, 2 min and 2 hours, or 2 min and 0.5 hours or between about 2 min and less than 5 hours, less than 3 hours, less than 2 hours or less than 1 hour at a constant temperature between 20-37° C., preferably between 22-28° C., more preferably between 24-26° C. In certain embodiments the reaction mixture has a volume of 300 μl or less. In certain embodiments multiple reaction mixtures are analysed in parallel, such as 12 or more, 24 or more, 36 or more, 72 or more or 96 or more, preferably in a volume of 300 μl or less

The reaction solution used in the method according to the invention (for both, the hydrophilic substrate HPTS ester and the lipophilic substrate 4-MU ester) further comprises a buffer having a pH of about pH 4 to about pH 8. Preferably the method is performed using a buffer having a pH of about pH 5 to about pH 7.5, more preferably about pH 5.5 to about pH 7.5. The person skilled in the art will understand that the pH of the buffer is within its buffering range when used in the method of the invention. In principle any buffer known in the art can be used, provided that is has a buffering range within about pH 4 to about pH 8. The buffer may comprise a single buffer substance or may be a multiple component buffer. Multiple component buffers typically have a broader buffering range. For example the buffer may comprise one or more buffer substances selected from the group consisting of a formic acid, acetic acid, lactic acid, citric acid, malic acid, maleic acid, glycine, glycylglycine, succinic acid, TES (2-{[tris(hydroxyme-thyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄. Preferably the buffer is a phosphate buffer (Na₂HPO₄ and NaH₂PO₄), a Tris buffer or a HEPES buffer. In certain embodiments, the buffer has a concentration of about 50 to 400 mM, preferably about 50 to 300 mM more preferably about 50 to 200 mM.

The buffer may further be a multi-component buffer comprising more than one buffer substance with overlapping buffering ranges in order to have a broader buffering range. The buffer may, e.g., comprise two, three, four, five or more buffering substances, preferably two or more buffering substances, more preferably three or more buffering substances. For example, the multi-component buffer may comprise two to four buffering substances, three to four buffering substances, more preferably 3 buffering substances. In certain embodiments, the multi-component buffer comprises at least three buffer substances with overlapping buffering ranges, preferably comprising at least one of Tris, MES and/or acetic acid, preferably acetic acid, MES and Tris at a ratio of 1:1:2.

Since the assay turned out to be sensitive to ionic strength it is important for the design of a suitable multi-component buffer not only that it comprises buffer substances with overlapping buffer ranges, but that the buffer only moderately changes (less than 15% preferably even less than 10%) ionic strength at different pH (range pH 4-8) (Ellis KJ, Morrisson JF, 1982. Methods in Enzymology, 87: 405-426). For example, the AMT buffer comprising acetic acid, MES and Tris allows the use of the buffer at different pH with only moderately affecting ionic strength, e.g., to identify conditions, including pH conditions that reduce hydrolytic activity. This buffer further allows taking measurements at the pH of the sample to determine lipase activity at the specific conditions present in a sample as well as to compare lipase activity at different states during purification. The assay allows to further increase sensitivity by measuring the sample at pH optimum.

Thus, a multi-component buffer as disclosed herein allows for the use of a buffer with a variable pH from at least about pH 5 to at least about pH 7.5, or at least about pH 4 to at least about pH 8. Alternatively or in addition, the use of a buffer with different pH values between about pH 4 and about pH 8 affects the ionic strength of the buffer by less than 15%, preferably less than 10% or even less than 7.5% or less than 5%, such as from 0% to less than 15%, from 0% to less than 10%, from 0% to less than 7.5% or from 0% to less than 5%, or such as from 2% to less than 15%, from 2% to less than 10%, from 2% to less than 7.5% or from 2% to less than 5%. In one embodiment the use of a buffer with different pH values between about pH 4 and about pH 8 affects the ionic strength of the buffer by less than 15%, preferably less than 10% or even less than 7.5% or less than 5%, such as from 0% to less than 15%, from 0% to less than 10%, from 0% to less than 7.5% or from 0% to less than 5%, or such as from 2% to less than 15%, from 2% to less than 10%, from 2% to less than 7.5% or from 2% to less than 5%. The use of a multi-component buffer as disclosed herein further allows adjusting the pH of the buffer to the pH of the sample (without changing the buffer composition of the buffer). The use of a multi-component buffer as disclosed herein further allows adjusting the pH of the buffer to near the optimum of the at least one contaminating protein having lipase activity (thereby increasing sensitivity of the method) and/or comparing and identifying conditions that reduce hydrolytic activity.

The reaction solution may further comprise a non-buffering salt. In the present invention any salt that dissociates in water and has no buffering effect may be suitable for adjusting the ionic strength of the reaction solution. Examples for suitable salts are NaCl, KCl, or CaCl₂. In a certain example of the present invention, the non-buffering salt is selected from the group consisting of NaCl, KCl and CaCl₂, preferably the non-buffering salt is NaCl or KCl.

The concentration of the optional non-buffering salt may be in a range of about 100 mM to about 200 mM. In a certain embodiment of the present invention, the non-buffering salt has a concentration of about 100 mM to about 200 mM, preferably about 130 mM to about 170 mM, more preferably about 140 mm to about 150 mM in the reaction mixture. However, ionic strength in the reaction mix should not exceed a certain value due to negative impact on lipase activity. For example, the ionic strength of the optional non-buffering salt is preferably about 200 mM or less, about 190 mM or less, about 180 mM or less, about 170 mM or less, about 160 mM or less, or about 150 mM or less in the reaction mixture, such as from about 100 mM to about 200 mM, preferably about 130 mM to about 170 mM, more preferably about 140 mM to about 150 mM in the reaction mixture. In certain embodiments, the cumulative ionic strength of the ionic strength of the buffer and the non-buffering salt in the reaction mixture does not exceed about 450 mM. Accordingly, the cumulative ionic strength of the buffer and the non-buffering salt the reaction mixture may be about 450 mM or less, about 400 mM or less, about 380 mM or less, about 360 mM or less or about 350 mM or less. For example, the cumulative ionic strength of the buffer and the non-buffering salt the reaction mixture may be about 150 mM to about 450 mM or less, about 150 mM to about 400 mM or less, about 150 mM to about 380 mM or less, about 150 mM to about 360 mM or less or about 150 mM to about 350 mM or less.

The buffer according to step (b) of the methods of the invention and the buffer according to step (bi) of the methods of the invention may be the same or may be different. In a preferred embodiment the buffer according to step (b) of the methods of the invention and the buffer according to step (bi) of the methods of the invention are the same. Thus, the buffer components, the concentration(s) and the pH value or pH values in a range are the same. The same applies to the optional non-buffering salt, its presence and its concentration. This excludes other differences than the substrate specificity when detecting carboxylesterase activity and lipase activity.

The methods according to the present invention is suitable for detecting the fluorescence in a fluorescence spectrometer or a microplate spectrophotometer (preferably at λ_{Ex} 401-405 nm, λ_{Em} 510-516 nm for HPTS and at λ_{Ex} 330-340 nm, λ_{Em} 450 nm for 4-MU). Thus, the reaction mixture is contained (and preferably mixed) in a cuvette or a microtiter plate, preferably an at least 96-well microtiter plate for measurement. The method according to the present invention is therefore particularly suitable for high throughput analysis and/or automated analyses of samples. In certain embodiments, in the method according to the present invention at least 2, 3, 4, 5, 10 or more samples are analyzed simultaneously. Further, each sample is preferably measured at least in triplicates. Preferably, the method according to the invention is therefore performed using a microtiter plate having 96 wells or a multiple of 96 wells. Microtiter plates are not only be used for measuring hydrolysis in step (d) or step (di), but also for contacting the at least one sample with a reaction solution in step (d) or step (di) and incubating the sample with the substrate in the reaction mixture in step (c) or step (ci). Thus, in certain embodiment the samples are contacted, incubated and measured in a microtiter plate format having 96 wells or a multiple of 96 wells.

In certain embodiments, the sample is provided at about 30% (v/v) or less, preferably at about 25% (v/v) or less of the reaction mixture. Thus, the sample may be provided at about 20% (v/v) to about 30% (v/v) of the reaction mixture, preferably at about 20% (v/v) to about 25% (v/v) of the reaction mixture. Optionally the sample may be pre-diluted. The at least one sample comprising a recombinant protein of interest may be a harvested cell culture fluid (HCCF) or a cell lysate, an in-process control (IPC) sample, a drug substance sample or a drug product sample, preferably an IPC sample, a drug substance sample or a drug product sample. Preferably, contacting the at least one sample with a reaction solution to form a reaction mixture comprises mixing the at least one sample with the reaction solution to obtain a homogenous reaction mixture. This is preferably done by adding the smaller volume (typically the sample) first and adding the larger volume (typically the reaction solution) second. Preferably, the components of the reaction solution are added as a master mix, wherein the master mix may be prepared as a concentrate that is diluted to working concentration prior to addition to the sample.

The at least one sample may be a harvested cell culture fluid (HCCF) or a cell lysate, an in-process control (IPC) sample, a UF/DF sample, a drug substance sample or a drug product sample. The recombinant protein of interest in the sample for detecting carboxylesterase activity and optionally lipase activity is preferably a therapeutic protein, such as an antibody, an antibody fragment, an antibody derived molecule, a fusion protein (e.g., an Fc fusion protein), a growth factor, a cytokine or a hormone, preferably an antibody, an antibody fragment, an antibody derived molecule or an Fc fusion protein. Thus, the recombinant protein of interest is preferably a secreted protein. The term “harvested cell culture fluid” or “HCCF” as used herein refers to the cell culture supernatant following harvest, i.e., following separation from the cells. According to the invention the recombinant protein of interest in the sample for detecting lipase activity is not a carboxylesterase or lipase and/or does not comprise carboxylesterase or lipase activity. Thus, any carboxylesterase or lipase activity detected in the at least one sample is contaminating carboxylesterase or lipase activity and/or derived from at least one contaminating host cell protein having carboxylesterase or lipase activity, such as CHO host cell proteins (CHOPs) derived from the CHO cell. Moreover, the recombinant protein of interest in the sample according to the methods of the present invention is not an esterase or hydrolase and/or does not comprise an esterase or hydrolase activity. In certain embodiments, the recombinant protein of interest is produced in a CHO cells and the at least one contaminating host cell protein is a CHO host cell protein (CHOP).

Thus, the method according to the invention can be advantageously used for detecting carboxylesterase or lipase activity by measuring hydrolysis in a sample comprising an antibody, an antibody fragment, an antibody derived molecule or a fusion protein (e.g., an Fc fusion protein). Typically, an antibody is mono-specific, but an antibody may also be multi-specific. Thus, the method according to the invention may be used for samples comprising mono-specific antibodies, multi-specific antibodies, or fragments thereof, preferably of antibodies (mono-specific), bispecific antibodies, trispecific antibodies or fragments thereof, preferably antigen-binding fragments thereof. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R, anti-IL-36R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2, anti-IL-36R and anti-IgE antibodies.

The term “antibody”, “antibodies”, or “immunoglobulin(s)” as used herein relates to proteins selected from among the globulins, which are naturally formed as a reaction of the host organism to a foreign substance (=antigen) from differentiated B-lymphocytes (plasma cells). There are various classes of immunoglobulins: IgA, IgD, IgE, IgG, IgM, IgY, IgW. Preferably the antibody is an IgG antibody, more preferably an IgG1 or an IgG4 antibody. The terms immunoglobulin and antibody are used interchangeably herein. Antibody include monoclonal, monospecific and multi-specific (such as bispecific or trispecific) antibodies, a single chain antibody, an antigen-binding fragment of an antibody (e.g., an Fab or F(ab′)2 fragment), a disulfide-linked Fv, etc. Antibodies can be of any species and include chimeric and humanized antibodies. “Chimeric” antibodies are molecules in which antibody domains or regions are derived from different species. For example, the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues. Antibodies may be produced through chemical synthesis, via recombinant or transgenic means, via cell (e.g., hybridoma) culture, or by other means.

Typically antibodies are tetrameric polypeptides composed of two pairs of a heterodimer each formed by a heavy and a light chain. Stabilization of both the heterodimers as well as the tetrameric polypeptide structure occurs via interchain disulfide bridges. Each chain is composed of structural domains called “immunoglobulin domains” or “immunoglobulin regions” whereby the terms “domain” or“region” are used interchangeably. Each domain contains about 70-110 amino acids and forms a compact three-dimensional structure. Both heavy and light chain contain at their N-terminal end a “variable domain” or “variable region” with less conserved sequences which is responsible for antigen recognition and binding. The variable region of the light chain is also referred to as “VL” and the variable region of the heavy chain as “VH”.

Antigen-binding fragments include without being limited thereto e.g. “Fab fragments” (Fragment antigen-binding=Fab). Fab fragments consist of the variable regions of both chains, which are held together by the adjacent constant region. These may be formed by protease digestion, e.g. with papain, from conventional antibodies, but similarly Fab fragments may also be produced by genetic engineering. Further antibody fragments include F(ab′)2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to ink the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Thus, antibody fragments and antigen-binding fragments further include Fv-fragments and particularly scFv.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucine-zipper domains. However, there are also strategies in which the interaction between the VH/VL regions of the scFv is used for the multimerisation (e.g. dia-, tri- and pentabodies). By diabody the skilled person means a bivalent homodimeric scFv derivative. The shortening of the linker in a scFv molecule to 5-10 amino acids leads to the formation of homodimers in which an inter-chain VH/VL-superimposition takes place. Diabodies may additionally be stabilized by the incorporation of disulphide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a linker region. Examples of minibody-antibody proteins are known from the prior art.

By triabody the skilled person means a: trivalent homotrimeric scFv derivative. ScFv derivatives wherein VH-VL is fused directly without a linker sequence lead to the formation of trimers.

The skilled person will also be familiar with so-called miniantibodies which have a bi-, tri- or tetravalent structure and are derived from scFv. The multimerisation is carried out by di-, tri- or tetrameric coiled coil structures. In a preferred embodiment of the present invention, the gene of interest is encoded for any of those desired polypeptides mentioned above, preferably for a monoclonal antibody, a derivative or fragment thereof.

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

Antibodies comprising an antigen-binding fragment and an Fc region may also be referred to as full-length antibody. Full-length antibody may be mono-specific and multispecific antibodies, such as bispecific or trispecific antibodies.

Preferred therapeutic antibodies according to the invention are multispecific antibodies, particularly bispecific or trispecific antibodies. Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge. Trispecific antibodies are monoclonal antibodies which combine three antigen-binding specificities. They may be build on bispecific-antibody technology that reconfigures the antigen-recognition domain of two different antibodies into one bispecific molecule. For example, trispecific antibodies have been generated that target CD38 on cancer cells and CD3 and CD28 on T cells. Multispecific antibodies are particularly difficult to product with high product quality.

Another preferred therapeutic protein is a fusion protein, such as an Fc-fusion protein. Thus, the invention can be advantageously used for production of fusion proteins, such as Fc-fusion proteins. Furthermore, the method of increasing protein producing according to the invention can be advantageously used for production of fusion proteins, such as Fc-fusion proteins.

The effector part of the fusion protein can be the complete sequence or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG2. Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising e.g. the N-linked glycosylation site.

The recombinant protein of interest of the present invention is produced in a eukaryotic cell in cell culture. Preferably, the eukaryotic cell used for producing the recombinant protein of interest is a yeast cell (e.g., Saccharomyces, Kluyveromyces) or a mammalian cell (e.g., hamster or human cells). Yeast cells can be, without being limited thereto Saccharomyces cerevisiae, Pichia pastoris, Kluyveromyces lactis or marxianus. The mammalian cell is preferably a CHO cell, a HEK 293 cell or a derivative thereof. HEK293 cells include without being limited thereto HEK293 cells, HEK293T cells, HEK293F cells, Expi293F cells or derivatives thereof. Commonly used CHO cells for large-scale industrial production are often engineered to improve their characteristics in the production process, or to facilitate selection of recombinant cells. Such engineering includes, but is not limited to increasing apoptosis resistance, reducing autophagy, increasing cell proliferation, altered expression of cell-cycle regulating proteins, chaperone engineering, engineering of the unfolded protein response (UPR), engineering of secretion pathways and metabolic engineering.

Preferably, CHO cells that allow for efficient cell line development processes are metabolically engineered, such as by glutamine synthetase (GS) knockout and/or dihydrofolate reductase (DHFR) knockout to facilitate selection with methionine sulfoximine (MSX) or methotrexate, respectively.

Preferably, the CHO cell used for producing the recombinant protein of interest is a CHO-DG44 cell, a CHO-K1 cell, a CHO-DXB11 cell, a CHO-S cell, a CHO glutamine synthetase (GS)-deficient cell or a derivative ofany of these cells.

Non-limiting examples of mammalian cells which can be used in the meaning of this invention are also summarized in Table A. However, derivatives/progenies of those cells, other mammalian cells, including but not limited to human, mice, rat, monkey, and rodent cell lines, can also be used in the present invention, particularly for the production of biopharmaceutical proteins, such as the recombinant protein of interest.

TABLE A
Exemplary mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 | ATCC CRL-8544 |
| derivative) | |
| CHO | ECACC No. 8505302 |
| CHO wild type | ECACC 00102307 |
| CHO-K1 | ATCC CCL-61 |
| CHOZN ® | Merck SAFC |
| | GS−/− and DHFR−/− |
| CHO-DUKX | ATCC CRL-9096 |
| (=CHO duk⁻, | |
| CHO/dhfr^{-,}, CHO-DXB11) | |
| CHO-DUKX 5A-HS-MYC | ATCC CRL-9010 |
| CHO-DG44 | Urlaub G, et al., 1983. Cell. 33: 405-412. |
| CHO Pro-5 | ATCC CRL-1781 |
| CHO-S | Life Technologies A1136401; CHO-S is |
| | derived from CHO as disclosed in Deaven |
| | LL and Petersen DF, 1973, Chromosoma |
| | (Berl.) 41: 129-144; and Lewis NE et |
| | al., 2013, Nature Biotechnology 31: |
| | 759-765 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J, et al., 2011. BMC Proc. |
| | 5(Suppl 8): P133. |
| PER.C6 ® | Pau et al., 2001. Vaccines. 19: 2716-2721. |
| H4-II-E | ATCC CRL-1548 |
| | ECACC No. 87031301 |
| | Reuber, 1961. J. Natl. Cancer Inst. 26: |
| | 891-899. Pitot HC, et al., 1964. Natl. Cancer |
| | Inst. Monogr. 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

The recombinant protein of interest of the method of the invention is produced in eukaryotic cells in cell culture. Following expression, the recombinant protein is harvested and further purified. The recombinant protein of interest may be recovered from the culture medium as a secreted protein in the harvested cell culture fluid (HCCF) or from a cell lysate (i.e., the fluid containing the content of a cell lysed by any means, including without being limited thereto enzymatic, chemical, osmotic, mechanical and/or physical disruption of the cell membrane and optionally cell wall) and purified using techniques well known in the art. The samples obtained and/or analyzed at the various steps of purification are also referred to as in-process control (IPC) samples or process intermediates. The harvest typically includes centrifugation and/or filtration, such as to produce a harvested cell culture fluid or cell lysate, preferably harvested cell culture fluid. Thus, the harvested cell culture fluid or the cell lysate may also be referred to as clarified harvested cell culture fluid or clarified cell lysate. It does not contain living cells and cell debris as well as most cell components have been removed. Clarified typically means centrifugation or filtration, preferably filtration. Further process steps may include affinity chromatography, particularly Protein A column chromatography for antibodies or Fc-containing proteins, to separate the product from contaminants. Further process steps may include acid treatment to inactivate viruses, clarifying the product pool by depth filtration, preferably following acid treatment, to remove cell contaminants, such as HCPs and DNA. Further process steps may include in this order or any other order as may be appropriate in the individual case: ion exchange chromatography, particularly anion exchange chromatography to further remove contaminating cell components and/or cation exchange chromatography to remove product related contaminants, such as aggregates. Further, preferably following process steps may include nanofiltration to further remove viruses and ultrafiltration and diafiltration to concentrate the recombinant protein of interest and to exchange buffer, respectively.

Since carboxylesterase and lipase activity is associated with host cell protein contaminants, the method according to the present invention may be particularly useful for analyzing process intermediates after (preferably before and after) purification steps that remove HCPs in order to adapt the relevant step to more efficiently remove carboxylesterase and/or lipase activity in the process intermediates, such as before and after affinity chromatography, before and after depth filtration in combination with acid treatment and/or before and after anion exchange chromatography. In some embodiments the method comprises obtaining at least one sample after affinity chromatography, and/or after depth filtration in combination with acid treatment (or after acid treatment and/or after depth filtration) and/or after ion exchange chromatography, such as anion exchange chromatography and/or cation exchange chromatography, preferably anion exchange chromatography. In some embodiments the method comprises obtaining at least one sample before and after affinity chromatography and/or before and after depth filtration in combination with acid treatment (or before and after acid treatment and/or before and after depth filtration) and/or before and after ion exchange chromatography, such as anion exchange chromatography and/or cation exchange chromatography, preferably anion exchange chromatography. The person skilled in the art will know that the sample obtained after a certain method step may be the same as the sample obtained before the following method step, such as the sample obtained after affinity chromatography (e.g., Protein A chromatography) may be the same sample as the sample before acid treatment (or before depth filtration in combination with, i.e., following, acid treatment). As explained above, due to the broad buffering range of the buffer, even lipase activity in samples having different pH values can be compared using the method according to the invention. Other samples that may be analyzed using the method according to the invention are samples after ultrafiltration/diafiltration (UF/DF samples) drug substance or drug product samples. Drug substance or drug product samples comprise formulation buffer and therefore often contain polysorbate. At very high concentrations polysorbate can inhibit the hydrolysis reaction of the lipophilic substrate 4-MU ester due to competition with the substrate. However, due to the sensitivity of the lipophilic substrate 4-MU ester assay, at typical concentrations of 0.4 to 0.8 mg/mi polysorbate, lipase activity can also be determined in a drug substance or drug product sample diluted as explained above in the final reaction mixture. The hydrophilic substrate HPTS ester assay is slightly more sensitive to polysorbate in the reaction and polysorbate should not exceed 50 μg/ml. Polysorbate is typically added following ultrafiltration/diafiltration (following capture and polishing steps) to form the drug substance. Thus, alternatively UF/DF samples may be used for detecting carboxylesterase and/or lipase activity in the (final) purified product.

The method of manufacturing a recombinant protein of interest typically comprises an ultrafiltration diafiltration step using tangential flow filtration (TTF) to provide the purified antibody product pool. The purified recombinant protein of interest is buffer exchanged and further concentrated by ultrafiltration. Polysorbate is then added to the concentrated antibody comprising all other excipients of the formulation.

In one aspect a method of manufacturing a recombinant protein of interest is provided comprising the steps of (i) cultivating a eukaryotic cell expressing a recombinant protein of interest in cell culture; (ii) harvesting the recombinant protein of interest; (iii) purifying the recombinant protein of interest; (iv) optionally formulating the recombinant protein of interest into a pharmaceutically acceptable formulation suitable for administration; and (v) obtaining at least one sample comprising the recombinant protein of interest in steps (ii), (iii) and/or (iv); wherein the method further comprises detecting carboxylesterase activity in a sample obtained in step (v) (i.e., a sample comprising the recombinant protein of interest and at least one contaminating host cell protein) comprising: (a) providing the at least one sample obtained in step (v) comprising the recombinant protein of interest produced in a eukaryotic cell in cell culture and at the least one contaminating host cell protein; (b) contacting the at least one sample with a reaction solution comprising a hydrophilic substrate to form a reaction mixture, wherein the reaction solution comprises: (i) a buffer having a pH of about pH 4 to about pH 8, (ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and (iv) optionally a non-buffering salt; (c) incubating the sample and the substrate in the reaction mixture; and (d) detecting carboxylesterase activity of the at least one contaminating host cell protein by measuring hydrolysis of the substrate HPTS ester and detecting the fluorescence intensity of the released chromophore HPTS; wherein the method optionally further comprises: (bi) contacting the at least one sample comprising a recombinant protein of interest produced in a eukaryotic cell in cell culture and at least one contaminating host cell protein of step (a) in a separate reaction set-up with a reaction solution comprising a hydrophilic substrate to form a reaction mixture, wherein the reaction solution comprises; (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester (substrate 4-MU ester), (iii) optionally a non-buffering salt, and (iv) a non-denaturing detergent not having an ester-bond, wherein the detergent is a non-ionic or zwitterionic detergent, (ci) incubating the sample and the substrate in the reaction mixture; (di) detecting lipase activity of the at least one contaminating host cell protein by measuring hydrolysis of the 4-MU ester and detecting the fluorescence intensity of the released chromophore 4-MU; optionally measuring hydrolysis by detecting the fluorescence intensity of the released chromophore HPTS or 4-MU over time, while incubating the sample and the substrate in the reaction mixture according to step (c) or (ci), respectively. Thus, in certain embodiments the method further comprises detecting lipase activity in a sample (obtained in step (v)) comprising: (bi) contacting the at least one sample comprising a recombinant protein of interest produced in a eukaryotic cell in cell culture and at least one contaminating host cell protein of step (a) in a separate reaction set-up with a reaction solution comprising a hydrophilic substrate to form a reaction mixture, wherein the reaction solution comprises; (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester (substrate 4-MU ester), (iii) optionally a non-buffering salt, and (iv) a non-denaturing detergent not having an ester-bond, wherein the detergent is a non-ionic or zwitterionic detergent, (ci) incubating the sample and the substrate in the reaction mixture; (di) detecting lipase activity of the at least one contaminating host cell protein by measuring hydrolysis of the 4-MU ester and detecting the fluorescence intensity of the released chromophore 4-MU; optionally measuring hydrolysis by detecting the fluorescence intensity of the released chromophore HPTS or 4-MU over time, while incubating the sample and the substrate in the reaction mixture according to step (c) or (ci), respectively. In certain embodiments the recombinant protein of interest is a therapeutic protein, such as an antibody, an antibody fragment, an antibody derived molecule (e.g., scFv, bi- or multi-specific antibodies) or a fusion protein (e.g., an Fc fusion protein). The person skilled in the art will understand that the steps of (i) cultivating a eukaryotic cell; (ii) harvesting the recombinant protein of interest; (iii) purifying the recombinant protein of interest; and (iv) optionally formulating the recombinant protein of interest may comprise several substeps. For examples step (iii) purifying the recombinant protein of interest may comprise the substeps of purifying the recombinant protein of interest using affinity chromatography, acid treatment, depth filtration, and/or ion exchange chromatography.

In certain embodiments the method of manufacturing a recombinant protein of interest according to the invention comprises obtaining at least one sample comprising the recombinant protein of interest in a step of harvesting the recombinant protein of interest (in step (ii)), wherein the sample is a harvested cell culture fluid (HCCF) or a cell lysate; in a step of purifying the recombinant protein of interest (in step (iii)), wherein the sample is an in-process control (IPC) sample; and/or in the optional step of formulating the recombinant protein of interest into a pharmaceutically acceptable formulation suitable for administration (in step (iv)), wherein the sample is a drug substance sample or a drug product sample. Preferably, the method of manufacturing a recombinant protein of interest according to the invention comprises obtaining at least one sample comprising the recombinant protein of interest in step (iii), wherein the sample is an in-process control (IPC) sample, such as comprising obtaining at least one sample after affinity chromatography, after depth filtration following acid treatment (or after acid treatment and/or after acid treatment), and/or after ion exchange chromatography, preferably anion exchange chromatography or cation exchange chromatography. More preferably the method comprises obtaining at least one sample before and after affinity chromatography, before and after depth filtration following acid treatment (or before and after acid treatment and/or before and after acid treatment), and/or before and after ion exchange chromatography, preferably anion exchange chromatography or cation exchange chromatography. Other samples that may be analyzed using the method according to the invention are samples after ultrafiltration/diafiltration (UF/DF samples), drug substance or drug product samples. Thus, in certain embodiments, the method comprises obtaining at least one sample comprising the recombinant protein of interest in step (ii), wherein the sample is a harvested cell culture fluid (HCCF) or a cell lysate; and/or in step (iii), wherein the sample is an in-process control (IPC) sample; and/or in step (iv), wherein the sample is a UF/DF sample, a drug substance sample or a drug product sample; preferably comprising obtaining at least one sample comprising the recombinant protein of interest produced in a eukaryotic cell in cell culture and at least one contaminating host cell protein in step (iii), comprising obtaining at least one sample before and after affinity chromatography, before and after acid treatment, before and after depth filtration, and/or before and after ion exchange chromatography, preferably anion exchange chromatography or cation exchange chromatography. The step of detecting carboxylesterase or lipase activity in a sample comprising the recombinant protein of interest is performed according to and as specified in the method for detecting lipase activity as described herein.

In yet another aspect the invention relates to a use of a hydrophilic substrate HPTS ester and/or a lipophilic substrate 4-MU ester as a substrate for detecting in an assay carboxylesterase and/or lipase activity of at least one contaminating host cell protein in a sample comprising a recombinant protein of interest produced in a eukaryotic cell in cell culture, wherein the hydrophilic substrate is a saturated unbranched-chain fatty acid (C6-C12) ester of 1-hydroxypyrene-3,6,8-trisulfonic acid or a salt thereof (substrate HPTS ester), and wherein the lipophilic substrate is a saturated unbranched-chain fatty acid (C6-C16) 4-MU ester. Preferably, the recombinant protein is produced in a CHO cell and the at least one contaminating host cell protein is a CHO host cell protein (CHOP). In one embodiment the use is a use of a hydrophilic substrate HPTS ester as a substrate for detecting in an assay carboxylesterase activity of at least one contaminating host cell protein in a sample comprising a recombinant protein of interest produced in a eukaryotic cell in cell culture, wherein the hydrophilic substrate is a saturated unbranched-chain fatty acid (C6-C12) ester of 1-hydroxypyrene-3,6,8-trisulfonic acid or a salt thereof (substrate HPTS ester). In a preferred embodiment the use is a use of a hydrophilic substrate HPTS ester and a lipophilic substrate 4-MU ester as a substrate for detecting in an assay carboxylesterase and lipase activity of at least one contaminating host cell protein in a sample comprising a recombinant protein of interest produced in a eukaryotic cell in cell culture, wherein the hydrophilic substrate is a saturated unbranched-chain fatty acid (C6-C12) ester of 1-hydroxypyrene-3,6,8-trisulfonic acid or a salt thereof (substrate HPTS ester), and wherein the lipophilic substrate is a saturated unbranched-chain fatty acid (C6-C16) 4-MU ester. For the components of the reaction solution, the samples and the conditions, the same applies as specified above for the method of the invention.

### A Kit for Determining Contaminating Lipase Activity by Measuring Hydrolysis in a Sample

Also provided is a kit for determining contaminating carboxylesterase and/or lipase activity in a sample comprising a recombinant protein of interest comprising: (i) a buffer having a pH of about pH 4 to about pH 8; and (ii) a hydrophilic substrate and a lipophilic substrate, wherein (a) the hydrophilic substrate is a saturated unbranched-chain fatty acid (C6-C12) ester of 1-hydroxypyrene-3,6,8-trisulfonic acid or a salt thereof (substrate HPTS ester); and (b) the lipophilic substrate comprises the chromophore 4-methylumbelliferyl (4-MU) in the form of a 4-MU ester, wherein the 4-MU ester is a saturated unbranched-chain fatty acid (C6-C16) 4-MU ester (substrate 4-MU ester); and optionally (iii) a non-buffering salt; and/or (iv) a non-denaturing detergent not having an ester-bond, wherein the detergent is a non-ionic or zwitter-ionic detergent. The kit may further optionally comprise water for dilution. The hydrophilic substrate and the lipophilic substrate are used in a separate reaction set up. Only the reaction solution comprising the lipophilic substrate is prepared comprising the non-denaturing detergent. Thus, the reaction solution comprising the hydrophilic substrate is prepared not comprising the non-denaturing detergent and/or any detergent, particularly any detergent above its CMC. In certain embodiments the kit further comprises a manual with instructions that the reaction solution comprising the lipophilic substrate is prepared comprising the non-denaturing detergent and that the reaction solution comprising the hydrophilic substrate is prepared not comprising the non-denaturing detergent.

In one embodiment, the kit further comprises an internal standard that serves as positive control and/or allows to calculate relative values compared the internal standard, such as a commercially available carboxylase and/or lipase, e.g., porcine pancreatic lipase (PPL) crude extract. The kit may also comprise one or more microtiter plate having 96 wells or a multiple of 96 wells. The kit components may be provided as solutions and/or dry components, either separately or in a pre-mixed form. The buffer may be provided as a dry compound providing a buffer having a pH of about pH 4 to about pH 8 upon dilution or reconstitution.

In certain embodiments, the buffer, and the optional non-buffering salt for the reaction solution comprising the hydrophilic substrate or the buffer and the optional non-buffering salt and/or the optional detergent for the reaction solution comprising the lipophilic substrate are premixed as an assay buffer. Preferably, said assay buffer is at least about 3-fold concentrated or about 3-fold to about 5-fold concentrated relative to a final reaction mixture. Alternatively, the assay buffer is provided as a dry mixture. Such dry mixture may be reconstituted with water to provide said at least about 3-fold concentrated or 5-fold concentrated assay buffer relative to a final reaction mixture. In one embodiment, a dry mixture of the assay buffer is a lyophilized assay buffer. The substrate is provided separately to be added to the assay buffer before use to provide the reaction solution. Alternative the kit may comprise the buffer, the substrate and the optional non-buffering salt and/or the optional detergent, premixed as a master mix. The master mix may be adapted to be provided at about 80% (v/v) to about 70% (v/v) of the reaction mixture, preferably at about 80% to about 75% of the reaction mix. The assay buffer and the reaction solution are aqueous solutions.

For the components of the reaction solution the same applies as specified above for the method of the invention. The hydrophilic substrate HPTS ester is a saturated unbranched-chain fatty acid (C6-C12, preferably C8-C12) ester of 1-hydroxypyrene-3,6,8-trisulfonic acid or a salt thereof (substrate HPTS ester). Thus the hydrophilic substrate HPTS ester may be 1-hexanoyloxy-pyrene-3,6,8-trisulfonic acid or a salt thereof, 1-heptanoyloxy-pyrene-3,6,8-trisulfonic acid or a salt thereof, 1-octanoyloxy-pyrene-3,6,8-trisulfonic acid or a salt thereof (OPTS), 1-nonaoyloxy-pyrene-3,6,8-trisulfonic acid or a salt thereof, 1-decanoyloxy-pyrene-3,6,8-trisulfonic acid or a salt thereof, 1-undecanoyloxy-pyrene-3,6,8-trisulfonic acid or a salt thereof and 1-dodecanoyloxy-pyrene-3,6,8-trisulfonic acid or a salt thereof. In certain embodiments the hydrophilic substrate HPTS ester is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof. In certain preferred embodiments the hydrophilic substrate HPTS ester is 1-octanoyloxy-pyrene-3,6,8-trisulfonic acid or a salt thereof (OPTS), preferably 1-octanoyloxy-pyrene-3,6,8-trisulfonic acid trisodium salt.

The lipophilic substrate comprising the chromophore 4-MU is in the form of saturated unbranched-chain fatty acid (C6 to C16) 4-MU ester (substrate 4-MU ester), wherein the aliphatic chain of the saturated unbranched-chain fatty acid has from C6 to C16 carbon atoms or preferably from C8 to C12 carbon atoms. Thus, the lipophilic substrate 4-MU ester may be 4-methylumbelliferyl octanoate, 4-methylumbelliferyl nonanoate, 4-methylumbelliferyl decanoate (4-MUD), methylumbelliferyl undecanoate or methylumbelliferyl dodecanoate. In certain embodiments the lipophilic substrate is selected from the group consisting of 4-methylumbelliferyl octanoate, 4-methylumbelliferyl decanoate (4-MUD) and 4-methylumbelliferyl dodecanoate, in a preferred embodiment the lipophilic substrate is 4-MUD.

The kit may further comprise an organic solvent for dissolving the lipophilic substrate, or the lipophilic substrate is dissolved in an organic solvent. The lipophilic substrate may be provided as a dry substance and optionally an additional organic solvent or dissolved as a stock solution (such as a 100× stock solution relative to the concentration in the reaction mixture) in an organic solvent, such as dimethyl sulfoxide (DMSO) or dimethyl formamide (DMF), preferably DMSO. The kit may further comprise water for dissolving the hydrophilic substrate. The lipophilic substrate may be provided as a dry substance and should be frozen following solubilization in an aqueous solution such as water or an aqueous buffer.

Examples for suitable non-denaturing zwitter-ionic detergents and not having an ester bond (for the reaction solution comprising the lipophilic substrate) are without being limited thereto 3-[(3-cholamidopropyl)dimethylammonio]-1-propanesulfonate (CHAPS), 3-([3-Cholamidopropyl)dimethylammonio]-2-hydroxy-1-propanesulfonate (CHAPSO), CHAPS analogs (such as Big CHAP N,N-bis-(3-D-gluconamidopropyl)deoxycholamide), Zwittergent (different lengths, such as n-Dodecyl-N,N-dimethyl-3-ammonio-1-propanesulfonate (Zwittergent 3-12)) and 3-[N,N-Dimethyl(3-palmitoylaminopropyl)ammonio]-propanesulfonate or other amidosulfobetaine detergents. Examples for suitable non-denaturing non-ionic detergents are without being limited thereto pyranoside surfactants (such as Octyl β-D-glucopyranoside (OGP), Nonyl β-D-glucopyranoside, Dodecyl β-D-maltopyranoside (DDM) or Octyl β-D-thioglucopyranoside), polyoxyethylene (23) lauryl ether (Brij 35) or other Polyoxyethylene ether; saponins (e.g. Digitonin), octylphenoxy polyethoxyethanol (IGEPAL CA-630), poloxamer 188, 338, 407 or tergitol. In certain embodiments, the detergent is a non-denaturing non-ionic or zwitter-ionic detergent not having an ester-bond, preferably the detergent is not polyethylene glycol tert-octylphenyl ether (Triton X-100) and not polyethylene glycol nonylphenyl ether (NP-40). In a preferred embodiment the detergent is a non-denaturing non-ionic or zwitter-ionic detergent selected from the group consisting of CHAPS, CHAPSO, Zwittergent (such as Zwittergent 3-12) and a saponin, preferably CHAPS. The detergent is only needed in the reaction solution comprising the lipophilic substrate, while a detergent should be omitted from the reaction solution comprising the hydrophilic substrate.

In certain embodiments, the buffer comprises one or more buffer substances selected from the group consisting of formic acid, acetic acid, lactic acid, citric acid, malic acid, maleic acid, glycine, glycylglycine, succinic acid, TES (2-{[tris(hydroxyme-thyl)methyl]amino)ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄. In certain embodiments the buffer has a pH of about pH 4 to about pH 8, preferably the buffer has a pH of about pH 5 to about pH 7.5, more preferably the buffer has a pH of about pH 5.5 to about pH 7.5.

The buffer may comprise a single buffer substance or may be a multiple component buffer as specified above for the method according to the invention. The multi-component buffer may comprise more than one buffer substance with overlapping buffering ranges in order to have a broader buffering range. The buffer may, e.g., comprise two, three, four, five or more buffering substances, preferably two or more buffering substances, more preferably three or more buffering substances. For example the multi-component buffer may comprise two to four buffering substances, three to four buffering substances, more preferably 3 buffering substances. In certain embodiments, the multi-component buffer comprises at least three buffer substances with overlapping buffering ranges, preferably comprising at least one of Tris, MES and/or acetic acid, more preferably acetic acid, MES and Tris at a ratio of 1:1:2. In certain embodiments the buffer is a multi-component buffer having a buffering range from at least about pH 5 to at least about pH 7.5, preferably from at least about pH 4 to at least about pH 8. In one embodiment, the use of the multi-component buffer at different pH values between about pH 4 and about pH 8 affects the ionic strength of the buffer by less than 15%, preferably less than 10% or even less than 7.5% or less than 5%, such as from 0% to less than 15%, from 0% to less than 10%, from 0% to less than 7.5% or from 0% to less than 5%, or such as from 2% to less than 15%, from 2% to less than 10%, from 2% to less than 7.5% or from 2% to less than 5%. The optional non-buffering salt may be, e.g., NaCl, KCl and CaCl₂ and is preferably NaCl or KCl.

In view of the above, it will be appreciated that the invention also encompasses the following items:

Item 1 provides a method for detecting carboxylesterase activity of contaminating host cell protein in a sample comprising a recombinant protein of interest produced in a eukaryotic cell comprising (a) providing at least one sample comprising a recombinant protein of interest produced in a eukaryotic cell in cell culture and at least one contaminating host cell protein; (b) contacting the at least one sample with a reaction solution (comprising a hydrophilic substrate) to form a reaction mixture, wherein the reaction solution comprises: (i) a buffer having a pH of about pH 4 to about pH 8, (ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and (iii) optionally a non-buffering salt; (c) incubating the sample and the substrate in the reaction mixture; (d) detecting carboxylesterase activity of the at least one contaminating host cell protein by measuring hydrolysis of the substrate HPTS ester and detecting the fluorescence intensity of the released chromophore 1-hydroxypyrene-3,6,8-trisulfonic acid or a salt thereof (HPTS); and optionally measuring hydrolysis by detecting the fluorescence intensity of the released chromophore HPTS over time, while incubating the sample and the substrate in the reaction mixture according to step (c).

Item 2 specifies the method of item 1, wherein the sample and the substrate in the reaction mixture are incubated for any time period between 2 min and 5 hours, 2 min and 3 hours, 2 min and 2 hours, or 2 min and 0.5 hours.

Item 3 specifies the method of item 1 or 2 in that the reaction mixture has a volume of 300 μl and/or multiple reaction mixtures (such as 12 or more, 24 or more, 36 or more, 72 or more or 96 or more) are analysed in parallel, preferably in a volume of 300 μl or less.

Item 4 specifies the method of any one of the preceding items in that a portion of the at least one sample is used for each reaction mixture.

Item 5 specifies the method of any one of the preceding items in that the fluorescence of the released chromophore HPTS is determined using an excitation wavelength within a range of 401-405 nm and an emission wavelength within a range of 510-516 nm.

Item 6 specifies the method of any one of the preceding items, wherein the substrate HPTS ester is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof, preferably selected from the group consisting of 1-octanoyloxy-pyrene-3,6,8-trisulfonic acid (OPTS) trisodium salt, 1-nonaoyloxy-pyrene-3,6,8-trisulfonic acid trisodium salt and 1-decanoyloxy-pyrene-3,6,8-trisulfonic acid trisodium salt.

Item 7 specifies the method of any one of the preceding items in that the reaction solution (comprising the hydrophilic substrate) does not contain a detergent.

Item 8 specifies the method of any one of the preceding items in that the hydrophilic substrate HPTS ester is dissolved in an aqueous solution, preferably an aqueous solution not comprising DMSO or DMF

Item 9 specifies the method of any one of the preceding items in that the hydrophilic substrate HPTS ester is used at a final concentration in the reaction mixture of 50 μM or less, preferably of 30 μM or less.

Item 10 specifies that the method of any one of the preceding items in that the method further comprises: (bi) contacting the at least one sample comprising a recombinant protein of interest produced in a eukaryotic cell in cell culture and at least one contaminating host cell protein of step (a) in a separate reaction set-up with a reaction solution comprising a lipophilic substrate to form a reaction mixture, wherein the reaction solution comprises: (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester, (iii) optionally a non-buffering salt, and (iv) a non-denaturing detergent not having an ester-bond, wherein the detergent is a non-ionic or zwitterionic detergent; (ci) incubating the sample and the substrate in the reaction mixture of step (bi); and (di) detecting lipase activity of the at least one contaminating host cell protein by measuring hydrolysis of the substrate 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (ci).

Item 11 specifies the method of item 10, wherein the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate.

Item 12 specifies the method of item 10 or 11, wherein the substrate is provided at a final concentration of about 1 μM to about 1 mM in the reaction mixture.

Item 13 specifies the method of any one of items 10-12, wherein the substrate is provided as stock solution in an organic solvent, and wherein the stock solution is added at about 1% to about 5% (v/v) of the reaction mix and/or wherein the organic solvent is DMSO or DMF.

Item 14 specifies the method of any one of items 10-13, wherein the detergent has a final concentration in the reaction mixture above its critical micelle concentration in the reaction mixture.

Item 15 specifies the method of any one of items 10-14, wherein the detergent is selected from the group consisting of CHAPS, CHAPSO and Zwittergent, preferably CHAPS, and/or and is not polyethylene glycol tert-octylphenyl ether (Triton X-100) and not polyethylene glycol nonylphenyl ether (NP-40).

Item 16 specifies the method of any one items 10-15, wherein the detergent is CHAPS and is provided at a final concentration in the reaction mixture of about 8 mM to about 20 mM, preferably at about 8 mM to about 15 mM, more preferably at about 10 mM.

Item 17 specifies the method of any one of the preceding items, wherein the fluorescence intensity of the released chromophore HPTS and optionally 4-MU is detected without stopping hydrolysis of the hydrophilic substrate HPTS ester and optionally the lipophilic substrate 4-MU ester; and/or the sample and the substrate in the reaction mixture are incubated for any time period between 2 min and less than 5 hours, less than 3 hours, less than 2 hours, or less than 0.5 hours.

Item 18 specifies the method of any one of the preceding item, wherein the fluorescence intensity of the released chromophore HPTS and optionally 4-MU is detected over time and follows a pseudo-zero order reaction rate, and optionally wherein a reaction mixture that does not meet the requirement of pseudo-zero order reaction rate is excluded from analysis.

Item 19 specifies the method of any one of the preceding items, further comprising a step of (a) determining the rate of hydrolysis by detecting the fluorescence intensity of the released chromophore HPTS and optionally 4-MU as relative fluorescent units (RFU) and determining the amount of the released chromophore HPTS and optionally 4-MU (mol/s) by comparing it to a calibration curve generated by using defined concentrations of 4-MU, and/or (b) calculating a relative value compared to an internal standard.

Item 20 specifies the method of any one of the preceding items, wherein the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄.

Item 21 specifies the method of any one of the preceding items, wherein the buffer has a pH of about 5 to about 7.5, preferably the buffer has a pH of about 5.5 to about 7.5.

Item 22 specifies the method of any one of the preceding items, wherein the buffer is a multi-component buffer having a buffering range from at least about pH 5 to at least about pH 7.5, preferably from at least about pH 4 to at least about pH 8.

Item 23 specifies the method of item 22, wherein the multi-component buffer comprises at least three buffer substances with overlapping buffering ranges, preferably comprising at least one of Tris, MES and/or acetic acid.

Item 24 specifies the method of item 22 or 23, wherein the method comprises (a) the use of a buffer with a variable pH from at least about pH 4 to at least about pH 8; (b) the use of a buffer with different pH values between about pH 4 and about pH 8 thereby affecting the ionic strength by less than 15%, preferably less than 10%, preferably from 0% to less than 15%, from 0% to less than 10%, from 0% to less than 7.5% or from 0% to less than 5%; (c) adjusting the pH of the buffer to the pH of the sample; (d) adjusting the pH of the buffer to near the optimum of the at least one (contaminating) host cell protein having carboxylase and/or lipase activity; or (e) comparing and identifying conditions that reduce hydrolytic activity.

Item 25 specifies the method of any one of the preceding items, wherein at least 2, 3, 4, 5, 10 or more samples are analyzed simultaneously.

Item 26 specifies the method of any one of the preceding items, wherein the non-buffering salt is selected from the group consisting of NaCl, KCl and CaCl₂, preferably wherein the non-buffering salt is NaCl or KCl.

Item 27 specifies the method of any one of the preceding items, wherein the non-buffering salt has a concentration of about 100 mM to about 200 mM, preferably about 130 mM to about 170 mM, more preferably about 140 mM to about 150 mM in the reaction mixture.

Item 28 specifies the method of any one of the preceding items, wherein the ionic strength of non-buffering salt is about 200 mM or less in the reaction mixture, preferably about 150 mM or less in the reaction mixture, preferably about 100 mM to about 200 mM, preferably about 130 mM to about 170 mM, more preferably about 140 mM to about 150 mM in the reaction mixture.

Item 29 specifies the method of any one of the preceding items, wherein the cumulative ionic strength of the buffer and the non-buffering salt in the reaction mixture is about 450 mM or less, preferably about 400 mM, more preferably 350 mM or less in the reaction mixture.

Item 30 specifies the method of any one of the preceding items, wherein the fluorescence is detected using a fluorescence spectrometer or microplate spectrophotometer.

Item 31 specifies the method of item 30 in that the fluorescence is detected using a microplate spectrophotometer in the bottom read mode or the top read mode, preferably in the bottom read mode.

Item 32 specifies the method of any one of the preceding items, wherein the samples are contacted, incubated and measured in one or more microtiter plate having 96 wells or a multiple of 96 wells.

Item 33 specifies the method of any one of the preceding items in that a portion of the at least one sample is used for each reaction mixture.

Item 34 specifies the method of any one of the preceding items, wherein the at least one sample (or the portion of the at least one sample) is provided at about 20% to about 30% (v/v) of the reaction mixture, preferably at about 25% of the reaction mixture, optionally wherein the sample (or the portion of the at least one sample) is pre-diluted.

Item 35 specifies the method of any one of the preceding items, wherein the at least one sample is a harvested cell culture fluid (HCCF) or a cell lysate, an in-process control (IPC) sample, a UF/DF sample, a drug substance sample or a drug product sample.

Item 36 specifies the method of any one of the preceding items, wherein (a) the recombinant protein of interest is not a carboxylesterase or a lipase and/or an enzyme having carboxylesterase or lipase activity; and/or (b) the recombinant protein of interest is selected from the group consisting of an antibody, an antibody fragment, an antibody derived molecule and a fusion protein.

Item 37 specifies the method of any one of the preceding items, wherein the eukaryotic cell used for producing the recombinant protein of interest is a yeast cell or a mammalian cell, wherein the mammalian cell is preferably a CHO cell, a HEK 293 cell or a derivative thereof.

Item 38 specifies the method of any one of the preceding items, wherein the recombinant protein of interest is produced in a CHO cell and the at least one contaminating host cell protein is a CHO host cell protein (CHOP).

Item 39 provides a method of manufacturing a recombinant protein of interest comprising the steps of (i) cultivating a eukaryotic cell expressing a recombinant protein of interest in cell culture; (ii) harvesting the recombinant protein of interest; (iii) purifying the recombinant protein of interest; and (iv) optionally formulating the recombinant protein of interest into a pharmaceutically acceptable formulation suitable for administration; and (v) obtaining at least one sample comprising the recombinant protein of interest in steps (ii), (iii) and/or (iv); wherein the method further comprises detecting carboxylesterase activity in a sample comprising the recombinant protein of interest and at least one contaminating host cell protein comprising: (a) providing the at least one sample obtained in step (v) comprising the recombinant protein of interest produced in a eukaryotic cell in cell culture and at least one contaminating host cell protein; (b) contacting the at least one sample with a reaction solution comprising a hydrophilic substrate to form a reaction mixture, wherein the reaction solution comprises: (i) a buffer having a pH of about pH 4 to about pH 8, (ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and (iv) optionally a non-buffering salt; (c) incubating the sample and the substrate in the reaction mixture; and (d) detecting carboxylesterase activity of the at least one contaminating host cell protein by measuring hydrolysis of the substrate HPTS ester and detecting the fluorescence intensity of the released chromophore HPTS; wherein the method optionally further comprises detecting lipase activity in a sample comprising (bi) contacting the at least one sample comprising a recombinant protein of interest produced in a eukaryotic cell in cell culture and at least one contaminating host cell protein of step (a) in a separate reaction set-up with a reaction solution comprising a lipophilic substrate to form a reaction mixture, wherein the reaction solution comprises; (i) a buffer having a pH of about pH 4 to about pH 8, (ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester, (iii) optionally a non-buffering salt, and (iv) a non-denaturing detergent not having an ester-bond, wherein the detergent is a non-ionic or zwitterionic detergent, (ci) incubating the sample and the substrate in the reaction mixture; (di) detecting lipase activity of the at least one contaminating host cell protein by measuring hydrolysis of the substrate 4-MU ester and detecting the fluorescence intensity of the released chromophore 4-MU; optionally measuring hydrolysis by detecting the fluorescence intensity of the released chromophore HPTS or 4-MU over time, while incubating the sample and the substrate in the reaction mixture according to step (c) or (ci), respectively.

Item 40 specifies the method of manufacturing a recombinant protein of interest according to item 39 wherein the method comprises obtaining at least one sample comprising the recombinant protein of interest in in step (ii), wherein the sample is a harvested cell culture fluid (HCCF) or a cell lysate; in step (iii), wherein the sample is an in-process control (IPC) sample; and/or in step (iv), wherein the sample is UF/DF sample, a drug substance sample or a drug product sample.

Item 41 specifies the method of manufacturing a recombinant protein of interest according to item 39 or 40 to comprise obtaining at least one sample comprising the recombinant protein of interest in step (iii), wherein the sample is an in-process control (IPC) sample.

Item 42 specifies the method of manufacturing a recombinant protein of interest according to item 41, wherein the method comprises obtaining at least one sample after affinity chromatography, after depth filtration following acid treatment (or after acid treatment and/or after depth filtration), and/or after anion exchange chromatography, preferably obtaining at least one sample before and after affinity chromatography, before and after depth filtration following acid treatment (or before and after acid treatment and/or before and after depth filtration), and/or before and after anion exchange chromatography.

Item 43 specifies the method of manufacturing a recombinant protein of interest according to any one of items 39-42, comprising detecting carboxylesterase and optionally lipase activity in a sample comprising the recombinant protein of interest produced in a eukaryotic cell in cell culture and at least one contaminating host cell protein according to the method of any one of items 1-38.

Item 44 provides a kit for determining contaminating carboxylesterase and/or lipase activity in a sample comprising a recombinant protein of interest comprising: (i) a buffer having a pH of about pH 4 to about pH 8; and (ii) a hydrophilic substrate and a lipophilic substrate, wherein (a) the hydrophilic substrate is a saturated unbranched-chain fatty acid (C6-C12) ester of 1-hydroxypyrene-3,6,8-trisulfonic acid or a salt thereof (substrate HPTS ester); and (b) the lipophilic substrate comprises the chromophore 4-methylumbelliferyl (4-MU) in the form of a 4-MU ester, wherein the 4-MU ester is a saturated unbranched-chain fatty acid (C6-C16) 4-MU ester (substrate 4-MU ester); and optionally (iii) a non-buffering salt; and/or (iv) a non-denaturing detergent not having an ester-bond, wherein the detergent is a non-ionic or zwitter-ionic detergent.

Item 45 further specifies the kit according to item 44 in that the hydrophilic substrate HPTS ester is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof, preferably selected from the group consisting of 1-octanoyloxy-pyrene-3,6,8-trisulfonic acid (OPTS) trisodium salt, 1-nonaoyloxy-pyrene-3,6,8-trisulfonic acid trisodium salt and 1-decanoyloxy-pyrene-3,6,8-trisulfonic acid trisodium salt.

Item 46 further specifies the kit according to item 44 or 45 in that the lipophilic substrate 4-MU ester is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate.

Item 47 further specifies that kit according to any one of items 44-46 in that the kit further comprises one or more microtiter plate having 96 wells or a multiple of 96 wells.

Item 48 specifies the kit of any one of items 44-47 to further comprise an organic solvent for dissolving the lipophilic substrate 4-MU ester, preferably DMSO or DMF.

Item 49 specifies the kit of any one of items 44-48, wherein the detergent is not polyethylene glycol tert-octylphenyl ether (Triton X-100) and not polyethylene glycol nonylphenyl ether (NP-40) or wherein the detergent is a non-denaturing zwitter-ionic detergent selected from the group consisting of CHAPS, CHAPSO and Zwittergent, preferably CHAPS.

Item 50 specifies the kit of any one of items 44-49, wherein the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄.

Item 51 specifies the kit of any one of items 44-50, wherein the buffer has a pH of about 5 to about 7.5, preferably the buffer has a pH of about 5.5 to about 7.5.

Item 52 specifies the kit of any one of items 44-51, wherein the buffer is a multi-component buffer having a buffering range from at least about pH 5 to at least about pH 7.5, preferably from at least about pH 4 to at least about pH 8.

Item 53 specifies the kit of item 52, wherein the multi-component buffer comprises at least three buffer substances with overlapping buffering ranges, preferably comprising at least one of Tris, MES and/or acetic acid.

Item 54 specifies the kit of any one of items 44 to 53 in that the non-buffering salt is selected from the group consisting of NaCl, KCl and CaCl₂, preferably wherein the non-buffering salt is NaCl or KCl.

Item 55 provides a use of a hydrophilic substrate HPTS ester and a lipophilic substrate 4-MU ester as a substrate for detecting in an assay carboxylesterase and lipase activity of at least one contaminating host cell protein in a sample comprising a recombinant protein of interest produced in a eukaryotic cell in cell culture, preferably wherein the recombinant protein of interest is produced in a CHO cell and the at least one contaminating host cell protein is a CHO host cell protein (CHOP), wherein the hydrophilic substrate is a saturated unbranched-chain fatty acid (C6-C12) ester of 1-hydroxypyrene-3,6,8-trisulfonic acid or a salt thereof (substrate HPTS ester), and wherein the lipophilic substrate is a saturated unbranched-chain fatty acid (C6-C16) 4-MU ester.

Item 56 specifies a use of a hydrophilic substrate HPTS ester as a substrate for detecting in an assay carboxylesterase activity of at least one contaminating host cell protein in a sample comprising a recombinant protein of interest produced in a eukaryotic cell in cell culture, wherein the hydrophilic substrate is a saturated unbranched-chain fatty acid (C6-C12) ester of 1-hydroxypyrene-3,6,8-trisulfonic acid or a salt thereof (substrate HPTS ester), and preferably wherein the recombinant protein is produced in a CHO cell and the at least one contaminating host cell protein is a CHO host cell protein (CHOP).

Item 57 specifies the use of item 55 or 56 comprising detecting carboxylesterase and optionally lipase activity in a sample comprising the recombinant protein of interest produced in a eukaryotic cell in cell culture and at least one contaminating host cell protein according to the method of any one of items 1-38.

### EXAMPLES

A new assay was developed to characterize the activity of esterases that preferentially convert hydrophylic substrates (e.g. carboxylesterases). This assay was used to investigate various biotechnologically or biopharmaceutically relevant solutions which, in addition to a variety of stabilizing substances (buffer substances, additives for adjusting the ionic strength and detergents), may potentially contain trace amounts of enzyme impurities, such as lipases and other esterases. The following methods were used in the Examples.

### Fluorescence Spectroscopic Measurements

In order to be able to determine the enzymatic activity, a substrate, 1-octanoyloxy-pyrene-3,6,8-trisulfonic acid trisodium salt (OPTS), was chosen. Its hydrolytic product, 1-hydroxypyrene-3,6,8-trisulfonic acid trisodium salt (HPTS), can be detected by fluorescence spectroscopy. The change in the fluorescence signal over time is directly related to the change in the concentration of free fluorophore. The conversion was made possible by setting up a calibration curve with a subsequent linear fit (R²≥0.99). The experiments of the kinetics measurements with the OPTS/4-MUD substrates assay were performed in duplicates. The reaction mixture without the protein of interest, which served as the respective control, was subtracted from the protein-containing batch. The buffer used for OPTS measurements (hydrophilic substrate) contained 0.3 M HOAc, 0.3 M MES, 0.6 M TRIS and 0.6 M NaCl (4×AMT buffer; acetate, MES, TRIS). For 4-MUD substrate measurements (lipophilic substrate) 0.04 M CHAPS was added in addition. The buffer (AMT) was prepared in four-fold concentration (4×AMT) and was added at 25% (v/v) to the reaction mix. All measurements were carried out in multiwell plates with a preparation volume of 300 μL (75 μL 4×AMT buffer, 30 μL substrate, 75 μL enzyme-containing solution, x μL additive, 300 μL ad. H₂O) Unless otherwise stated, all fluorescence spectroscopic measurements were performed using 96-well, clear-bottom, non-binding plates (Greiner, Austria) on a SpectraMax M3 PlateReader (Molecular Device, USA) and recorded using SoftMax Pro 7.0.3 software (Molecular Device, USA). The OPTS assay measurements were typically taken at 25° C., λₑₓ=403 nm and λₑₘ=512 nm and the 4-MUD assay measurements were taken at 25° C., λₑₓ=330 nm and λₑₘ=450 nm. For evaluation, the detected fluorescence signal for a reaction mix without protein of interest (control) was subtracted from the reaction mix with protein of interest (e.g. DS). Hydrolysis of the substrate has been measured by detecting the fluorescence intensity of the released chromophore (4-MU or HPTS) immediately following mixing in real-time for a few minutes up to 5 hours depending on fluorescence intensity. The changes in the fluorescence signal over time were subsequently converted to hydrolytic activity using previously established calibration curves with a linear fit (R²≥0.99).

Setting Up a Calibration Curve with Linear Fit

To set up the calibration curve with linear fit, a serial 1:1 dilution of the free fluorophore HPTS in H₂O was prepared from 12 μM up to 7.8 nM. Each dilution of HPTS solution was pipetted into a plate before the AMT buffer was added (composition: 75 μL 4×AMT (pH var.), 75 μL HPTS solution, 150 μL H₂O). The calibration curve was separately generated for the different pH values of the AMT buffer used (pH values 4-8 in 0.5 step increments). The final concentration range of HPTS used for the calibration curve was 3 μM to 1.46 nM. After pipetting, the measurement was started directly.

Kinetics Measurement with OPTS/4-MUD Substrate and Antibody Preparations

The target concentration of OPTS was 25 μM and the target concentration of 4-MUD was 30 μM. For preparing a master mix, 4×AMT buffer was mixed with the appropriate volume of H₂O in a Falcon tube. The dialysed antibody (Ab) preparation samples (drug substance) or control solutions (75 μl) were transferred to 96 well plates. Optional inhibitors (10 μM orlistat, 1 mM PMSF and 10 mM EDTA) were added to the master mix comprising 4×AMT buffer and water and substrate (3 μl/sample 3 mM 4-MUD in DMSO or 30 μM/sample 250 μM OPTS in H₂O, aliquoted and frozen). The 4-MUD substrate was stored at a concentrated stock solution comprising 3 mM 4-methylumbelliferyl decanoate (4-MUD; FM25973, Carbosynth) in DMSO resulting in a 100× stock solution for use comprising 0.3 mM in DMSO. The 250 μM OPTS stock solution was prepared in H₂O, aliquoted and frozen until use. Although OPTS is more stable in DMSO compared to H₂O (prevents autohydrolysis), the substrate was diluted in water (or alternatively AMT buffer) to avoid interference of DMSO with the assay. The reaction was then started by adding the master mix (reaction solution) to the protein solution or control solution, respectively. The measurement was started immediately. The measuring time was 30 min and the measuring intervals 15 s. Stock solutions of the inhibitors were used as follows: 300 μM orlistat (Sigma-Aldrich, USA) in DMSO, 100 mM PMSF (Roth, Germany) in isopropanol and 500 mM EDTA (Sigma-Aldrich, USA) in H₂O.

To generate pH profiles of the protein solutions, kinetic measurements were carried out as described above at the indicated pH value. The added AMT buffer was adjusted to the pH indicated in a range of pH 4-8 (0.5 steps) and the kinetics were recorded for each pH value.

### Influence of Tropolone on the Fluorescence of Free Fluorophores

The influence of tropolone on the fluorescence of HPTS and 4-MU at pH=5.5 was tested using tropolone with a target concentration of 10 mM. 1:1 dilution series were performed for HPTS and 4-MU with a target concentration ranging from 3 μM to 96 nM. DMSO served as the control substance for tropolone. In addition, a 1:1 dilution series of tropolone with a target concentration of 10 mM-3.2 μM was performed.

### Example 1: Determination of λex and λem of the Free Fluorophore HPTS

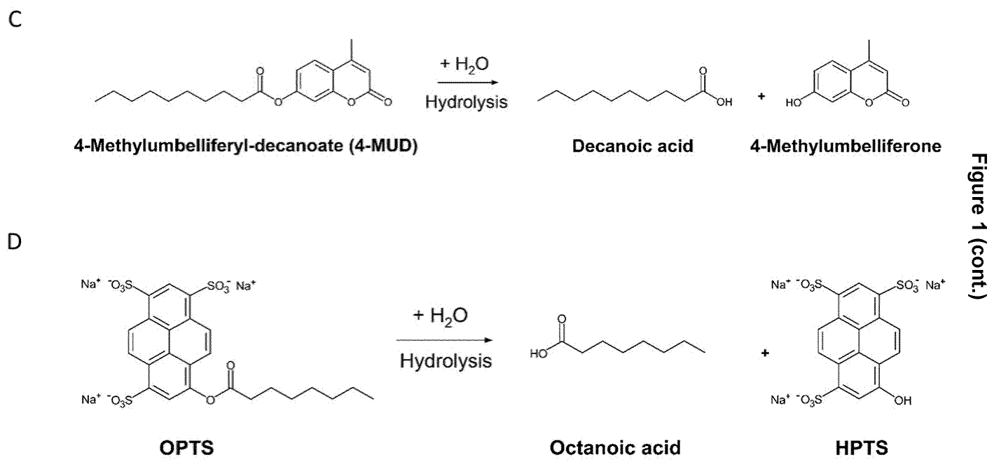

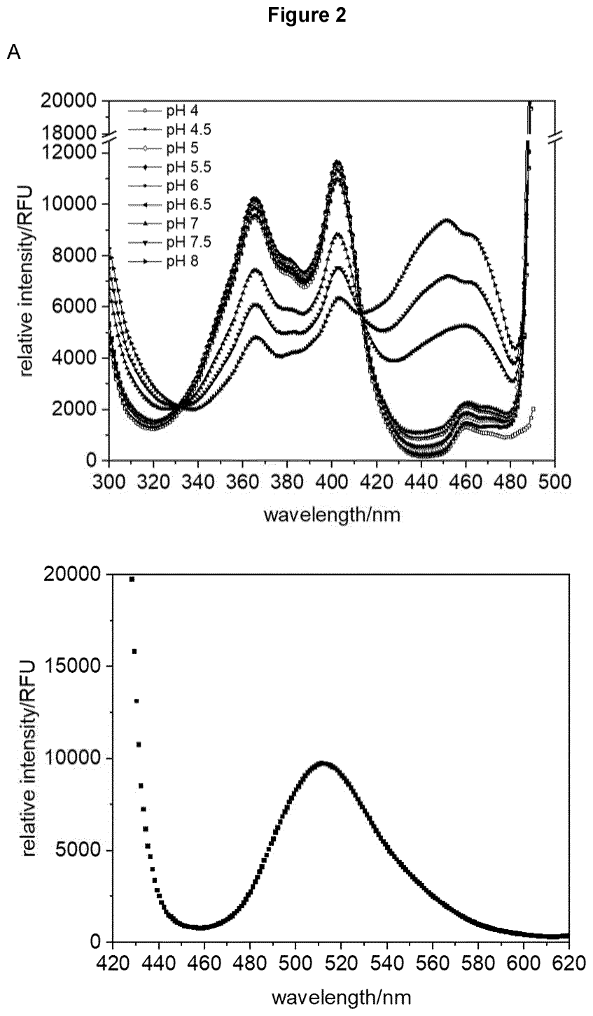

An aqueous solution of the free fluorophore (HPTS, Sigma-Aldrich) was prepared to record spectra for the excitation and emission wavelengths (see FIG. 2A). For the intended use, it was necessary that the fluorophore can be used in a pH range of preferably pH=4-8. For the determination of λex and λem, a final 1.5 μM HPTS aqueous solution was prepared with AMT buffer for pH values 4 to 8 (0.5 steps) by mixing the respective buffer with an aqueous HPTS solution in a plate (composition: 75 μL 4×AMT (pH var.), 75 μL 6 μM HPTS solution, 150 μL fully demineralised H₂O). To identify the possible influences of pH on the intensity of the resulting fluorescence signal of HPTS, spectra were recorded at varying excitation wavelength λex=300-500 nm and constant emission wavelength λem=520 nm and further spectra were recorded at constant excitation wavelength λex=415 nm and varying emission wavelength λem=420-620 nm.

The highest fluorescent signal was observed at λex=403 nm, the global maximum and no further change in intensity was observed for pH values 4-6.5. At pH>6.5 a sight loss of intensity was observed with increasing pH. However, the intensity gain at λex=403 nm provided a higher sensitivity even at pH=8 compared to the isosbestic point (λex=413 nm), where no pH-dependency was observed. For this reason, the excitation wavelength λex=403 nm was chosen for all further investigations. However, it is noted that the sensitivity decreases slightly with increasing pH (>6.5).

The emission maximum is at λem=512 nm, therefore, unless specifically mentioned, all measurements were recorded at this emission wavelength.

### Example 2: Calibration Curves Allow Quantification of Free Fluorophore

A calibration curves allow to correlate the recorded fluorescence signal with the fluorophore concentration in the solution. Thus, a change in the concentration of HPTS over time is based on the enzymatic activity and hence this can be calculated. To generate the calibration curve, a serial dilution of HPTS was prepared and measurements of these dilutions were made for each pH value of the AMT buffer (acetate, MES, TRIS). This buffer was used throughout the experiments, because it has a broad buffering range without major changes in ionic strength and osmolarity. These separate calibration curves further allow for correcting the pH-dependent changes of the fluorescence signal. In FIG. 2B at the top, the calibration lines are plotted for HPTS concentrations in the range of 1.46-3000 nM and at the bottom, the limit of the linear range is shown using the example of the calibration line at pH=7 for the HPTS concentration range of 93.75-6000 nM.

It can be seen that with increasing pH the slope of the calibration curve decreased. Concentrations at concentrations ≥3 μM showed a less perfect linear fit (R2 less than 0.99999), since the linear behaviour changes into an asymptotic behaviour. The deviation of the RFU value of the HPTS concentration of 6 μM from calibration curve (including 3 μM) is 7.4%. Thus, calibration curves up to 3 μM were used.

### Example 3: The Measuring Modes TR and BR—Influence on the Calibration Lines

The Multiwell PlateReader SpektraMax M3 (Molecular Devices, US), in which the fluorescence is not read out orthogonally to the excitation light, allows for two possible measurement modes, top read (TR) and bottom read (BR). It was therefore tested whether the measurement from above or below gives a different result using an aqueous dilution series of HPTS in the range of 1.5 μM to 1.46 nM, at pH 4-8 recorded with TR and BR.

For measurements in TR mode, multiwell plates with black, opaque bottom (96 well, black bottom plates; Greiner Bio-One, Austria) where used, whereas for measurements in BR mode, the plates have a special transparent bottom (96-well, clear bottom plates, Greiner Bio-One, Austria), which enables measurement from below. Further non-binding and medium binding plates (Greiner Bio-One) were tested, of which non-binding plates were found to be slightly better. BR measurement mode produced steeper calibration lines compared to TR measurements (data not shown), which means that smaller concentration changes can be calculated from BR measurements, corresponding to a higher sensitivity. In the following experiment measurements were performed in BR measurement mode for OPTS and 4-MUD using non-binding plates.

### Example 4: Hydrolysis of the Fluorogenic Substrate OPTS

The fluorogenic substrate OPTS (1-octanoyloxypyrene-3,6,8-trisulfonic acid, Sigma-Aldrich) consists of a tri-sulfonic acid-substituted pyrene esterified with caprylic acid. Due to the high solubility of OPTS in H₂O (≥2 mM), it represents a substrate for enzymes that convert hydrophilic substrates (such as carboxylesterases).

Stock solutions of the fluorogenic substrate in water were prepared. Aqueous solutions with an OPTS concentration ≥2.0 mM could be prepared. The solubility could be verified by light scattering experiments.

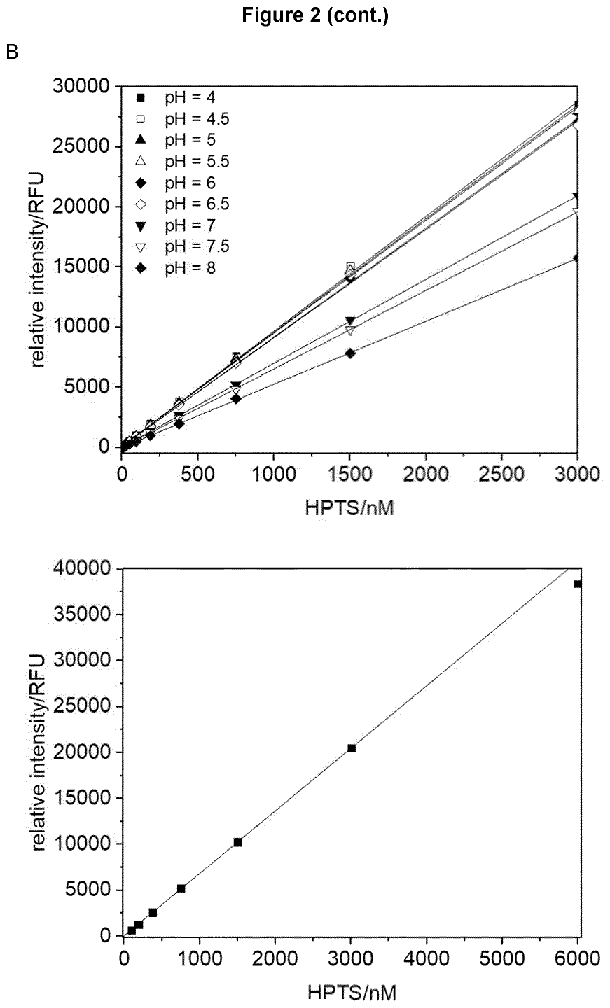

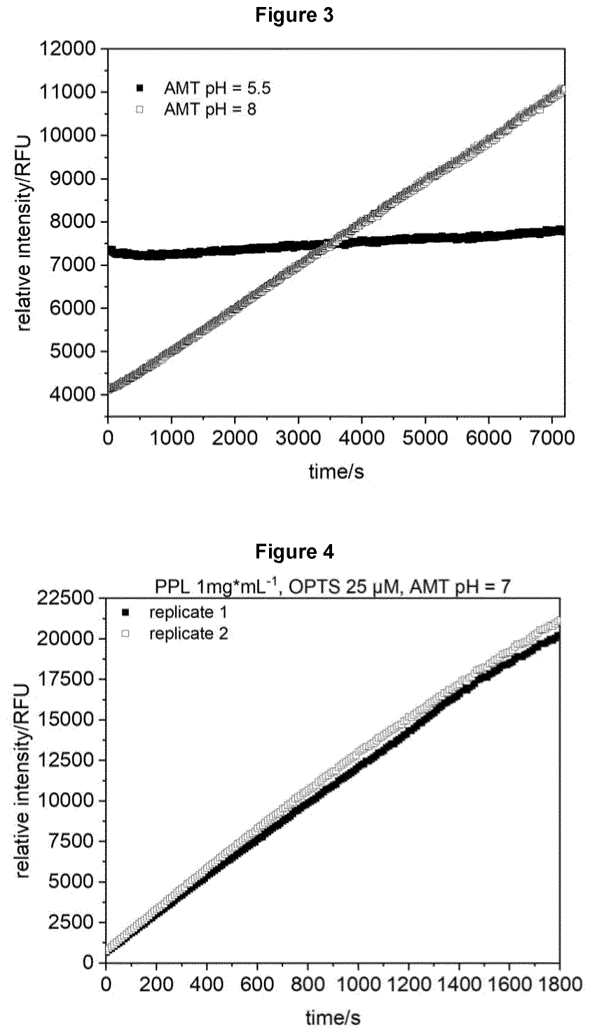

The fluorophore HPTS can be released from the substrate (here esterified caprylic acid) by enzymatic or chemical hydrolysis (acidic and alkaline hydrolysis). In order to assess the influence of pH on aqueous solutions of the substrate, 225 μL of a 33.3 μM OPTS solution was added to 75 μL each of AMT buffer (with pH=5.5 and pH=8) and the kinetics of fluorophore release were recorded for two hours. The results are shown in FIG. 3.

There is a clear difference in the slope of the fluorescence signals for the different pH values. Based on the recorded calibration curves the autohydrolysis rate at pH 5.5 was calculated as 1.58*10⁻⁷ units (μmol*min-1) and the autohydrolysis rate at pH 8.0 was calculated as 3.48*10⁻⁶ units. Thus, the hydrolysis rate at pH 8 is about 22-times higher compared to pH 5.5, showing that hydrolysis of the fluorogenic substrate can be observed over time, at basic pH, but only at a very low level at pH 5.5. Autohydrolysis increases with decreasing chain length and hence substrate HPTS esters from a saturated unbranched-chain fatty acid with less than six carbon atoms are expected to be unfavorable. Thus, a suitable substrate would be at least a C6 substrate HPTS ester, such as 1-hexanoyloxy-pyrene-3,6,8-trisulfonic acid trisodium salt, 1-heptanoyloxy-pyrene-3,6,8-trisulfonic acid trisodium salt or 1-octanoyloxy-pyrene-3,6,8-trisulfonic acid trisodium salt. Moreover, the pH should be maintained below pH 8.

### Example 5: Enzymatic Hydrolysis of the Fluorogenic Substrate OPTS

An aqueous solution of commercially purchased porcine pancreatic lipase, PPL, was prepared (Sigma-Aldrich, US; 100-500 Units/mg protein). According to the manufacturer, this powder mainly contains triacylglycerol lipase, but since it is a homogenised powder from freeze-dried porcine pancreas (crude extract), it is likely to further contain other carboxylesterases and lipases. These enzymes hydrolyse fatty acid esters and this extract was therefore selected for functional tests.

To record the reaction kinetics of the substrate OPTS with PPL, an aqueous 10 mg/mL PPL solution was prepared by adding 10 mg PPL to 1 mL H₂O. The suspension was vortexed for 2 min and centrifuged at 14,000×g for 5 min before the supernatant was removed.

A reaction mixture of 75 μL AMT buffer (pH=7), 165 μL H₂O and 30 μL of an aqueous 10 mg*mL-1 PPL solution was started with the addition of 30 μL of a 250 μM OPTS solution. The increase in fluorescence was recorded over 30 min with 15 s measuring intervals (n=2). As control (blank) served a preparation without PPL (only with H₂O). FIG. 4 shows the results (blank corrected) of two measurements. The data shown in FIG. 4 indicate that enzymes present in the PPL extract are capable of enzymatically hydrolysing OPTS to HPTS. The calculated reaction rate is 2.89±0.08*10-5 units (μmol*min-1).

To test for the concentration with maximum velocity vₘₐₓ, different concentrations of OPTS solutions were prepared by performing a serial 1:1 dilution series of an aqueous 10 mM OPTS solution. 30 μL of each of these solutions was added to 75 μL of AMT buffer (pH=7) and made up to 300 μL with H₂O. It was observed that at an OPTS concentration of <125 μM, there is no linearity between concentration and fluorescence intensity (R2≤0.999). All further measurements were therefore performed at an OPTS concentration of 25 μM.

### Example 6: Influence of Detergents on the Fluorogenic Substrate OPTS

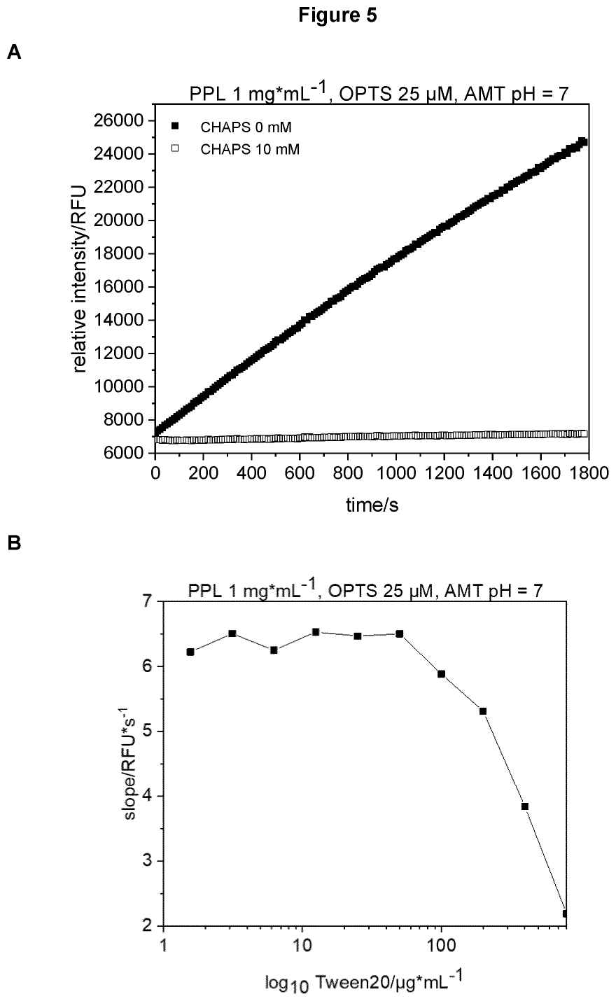

Detergents are added to a large number of samples that may be of interest for testing hydrolytic activity with the OPTS. In the complementary assay (for lipophilic substrates) using 4-MUD as substrate, detergents such as CHAPS are added above the critical micelle formation concentration for solubilization of the substrate and to open the id of the lipase that otherwise is covering the lipase's′ active site. For this purpose, CHAPS was added to a 25 μM OPTS solution with AMT buffer (pH=7) and 1 mg*mL-1 PPL at a final concentration of 10 mM. The second reaction batch was started without the addition of CHAPS. It was found that CHAPS has a negative influence on the enzymatic hydrolysis of OPTS by PPL (FIG. 5A). The activity of 2.53*10⁻⁵ units observed without CHAPS dropped to 6.16*10⁻⁷ in the presence of 10 mM CHAPS. The interference with enzymatic hydrolysis can have different reasons. Mixed micelles of CHAPS and OPTS may form, thus shielding the substrate and making it inaccessible to the active site of the enzymes (Lasic, Martin et al. 1989) or the free fluorophore HPTS may be affected by the presence of CHAPS. Experimental evidence indicated that the accessibility of the substrate OPTS is strongly reduced by CHAPS due to mixed micelle formation. Moreover, determining esterase activity in a buffer containing detergent is not desirable, because the assay is designed to measure enzymes that are able to turnover hydrophilic substrates rather than lipases (which may be more active in the presence of a detergent, due to the open-lid configuration). This is in clear contrast to the previously developed 4-MUD assay detecting lipase activity, which is more sensitive in the presence of CHAPS (WO 2022 049294 A1). It was further shown that Triton X-100 inhibited the assay, see FIG. 10 of WO 2022 049294 A1).

Further inhibition of the non-ionic detergent polysorbate 20 (Tween 20) was tested. Polysorbate 20 is one of the most often used detergents in biopharmaceutical products and is typically used above its CMC (CMC 10²-10³ μg/ml), typical concentrations used are 0.2-0.4 mg/ml. Target concentrations between 200 μg/mL and 0.098 μg/mL were tested using PPL. No inhibition was observed until 50 μg/ml at pH 7.0, but higher concentrations reduced the fluorescence signal (FIG. 5B). Subsequent experiment showed that this was due to a negative effect of Polysorbate 20 on the chromophore HPTS. Thus, polysorbate 20 at higher concentrations inhibits the fluorescence signal of HPTS slightly and hence should not exceed 100 μg/ml, preferably not 50 μg/ml. Drug substance samples analysed in this study had a polysorbate 20 concentration of 50 μg/ml or less following dilution in the reaction mixture.

The OPTS assay is intended for in-process control samples of recombinant protein of interest manufacture as a fast high-throughput assay for purification train development and optimization. Most in-process control samples do not contain detergents, such as polysorbate 20. Thus, this observation does not limit the applicability of the assay. The samples that may contain detergents are mainly the final drug substance and possibly the harvested cell culture fluid (HCCF) due to antifoaming agents. However, due to the high hydrolase activity in HCCF, samples would need to be diluted and hence potential antifoaming agents reach concentrations that would not interfere with the assay. With regard to the final product containing polysorbate, unless the polysorbate concentration is above the CMC following sample dilution in the reaction mix, this should not negatively affect the assay as in the present examples. Even a final polysorbate 20 concentration between 50 μg/ml and 100 μg/ml only slightly affects sensitivity of the assay. In case the polysorbate concentration in the reaction mixture (i.e., following dilution) is clearly above the CMC (such as above 100 μg/ml), UF/DF samples, i.e., prior to addition of polysorbate, may be used for determining hydrolytic activity using the OPTS assay. However, we note in this context that the assay is particularly useful for high throughput analysis of IPC samples in small volumes for process optimization, which typically do not contain polysorbate.

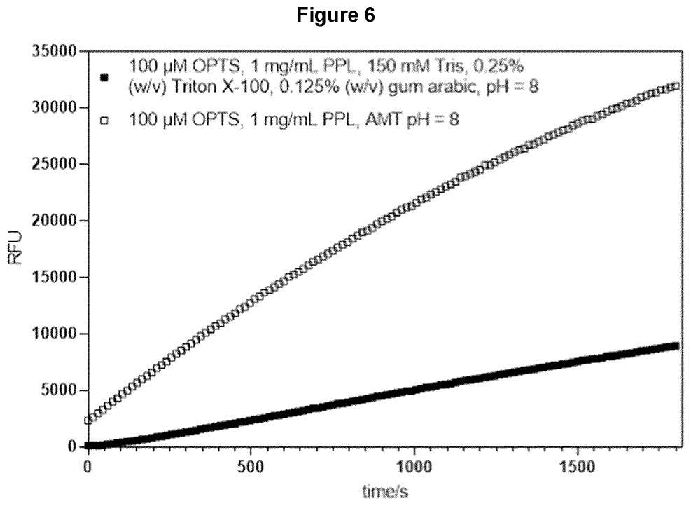

Finally, activity of the OPTS assay in a buffer as described in WO 2022/047416 A1 (page 37) was tested. For this purpose, a 150 mM Tris, 0.25% (w/v) Triton X-100, 0.125% (w/v) gum arabic (TTG buffer), pH=8 was tested comprising 1 mg*mL-1 PPL and a final OPTS concentration of 100 μM and compared to 100 μM OPTS, 1 mg/ml PPL in the AMT buffer (without detergent) at pH 8. The OPTS concentration was increased from 25 μM to 100 μM to match the substrate concentration of 100 μM 4-MU containing fluorogenic substrate used in WO 2022/047416 A1. Triton X-100 (MW=625 g/mol) has a critical micelle concentration (CMC) of 0.22 to 0.24 mM and hence 0.25% (w/v) Triton X-100 (4 mM) is well above its CMC. It was found that the TTG buffer had a negative influence on the enzymatic hydrolysis of OPTS by PPL (FIG. 6). In the buffer without detergent (AMT buffer) a 3-4 times faster enzyme-mediated hydrolysis of OPTS was observed compared to the TTG buffer comprising Triton X-100. Although the concentration of Triton X-100 was used above the CMC, the incomplete inhibition may be explained by the excess of OPTS potentially resulting in some substrate being accessible outside the formed micelles (autohydrolysis has been subtracted from the measured signal). With an aggregation number of about 80 for Triton X-100 (Stubidar N. et al., Micelles Determined by Light and Small-Angle X-Ray Scattering Techniques. In Mittal, K. L. (ads) Surfactants in Solution, Springer Boston, MA, pages 181-195) about 50 μM micelles are formed and hence a substrate concentration of 100 μM would be present in molar excess. Still, the buffer and conditions described in WO 2022/047416 A1 are not suitable for the OPTS assay of the present invention. Particularly, determining esterase activity in a buffer containing detergent (and particularly a detergent above its CMC) is not desirable. This assay is designed to measure enzymes that are able to turnover hydrophilic substrates rather than lipases, thus omitting a detergent is advantageous for detecting carboxylesterases and potentially further reduces background detection of lipase activity, which may be more active in the presence of a detergent (due to the open-id configuration and the accessibility of substrate within the micelles). The buffer used for measuring hydrolysis of hydrophilic substrates should therefore be free of detergent or at least not exceed the CMC of the detergent. Thus, the conditions described herein for the OPTS assay, especially omitting a detergent, render the assay specific for carboxylesterases acting on hydrophilic substrates.

The results show that the buffer described in WO 2022/047416 A1 cannot be used for the present OPTS buffer due to the presence of a detergent. Moreover, at pH 8.0 autohydrolysis is increased, resulting in reduced sensitivity of the assay and autohydrolysis was found to be more critical for OPTS compared to 4-MUD, particularly at pH 8.0 (see FIG. 3). Also most of the samples to be analysed using the OPTS assay are in the acidic rather than alkaline range.

### Example 7: Determination of Hydrolytic Activity in Antibody Solutions Using the OPTS Assay

The purpose of the OPTS assay is to characterise the activity of esterases (e.g. carboxylesterases) in samples comprising recombinantly produced proteins, such as antibodies. The tested samples were drug substance samples from 9 different antibody preparations, including IgG1 and IgG4 monoclonal antibodies as well as bispecific IgG-like formats, which were produced in CHO K1 or DG44 cells. The drug substance samples comprise, stabilizing substances (buffer substances, additives to adjust the ionic strength and detergents) and small amounts of contaminating host cell proteins that potentially exhibit hydrolase activity. In order to verify that enzymatic activity is involved, the antibody samples were thermally pre-treated at 50° C. in a ThermoMixer C (Eppendorf, Germany) and cooled to RT (passively) before kinetics measurements were performed as described. The activity in all samples decreased with increasing length of thermal incubation, which indicates enzymatic activity.

9 different antibody (Ab1-Ab9) samples were tested using the OPTS assay. As a control, measurements were performed with the respective same buffer solution as used in the antibody sample.

For some of the antibody solutions, controls showed slightly higher activity compared to the sample (data not shown) and these sample all contained histidine at about 25 mM. Since histidine has been reported previously to mediate hydrolytic activities in aqueous solutions similar to esterases (Delort, Nguyen-Trung et al. 2006, Mason, McCracken et al. 2010, Amano, Kobayashi et al. 2014, Xu, Chen et al. 2017), samples were dialysed against 0.002% NaCl using a two-step dialysis with a molecular weight cut-off (MWCO) of 20K. For preparative dialysis, the pre-wettened dialysis cassette was filled with 2 ml antibody solution and placed in a beaker filled with 1 L NaCl (0.002% w/v) solution with gentle stirring. After one hour the 1 L NaCl solution was replaced with fresh NaCl (0.002% w/v) solution and dialysed for another two hours with gentle stirring. For histidine concentrations of less than 200 μM, activity was found in the low 10⁻⁷ units range (data not shown) and hence the influence of this buffer component can be almost neglected. In the following the assay was performed using dialysed drug substance samples. The same solution (final solution outside the dialysis cassette) was used as control sample to be subtracted from the test sample.

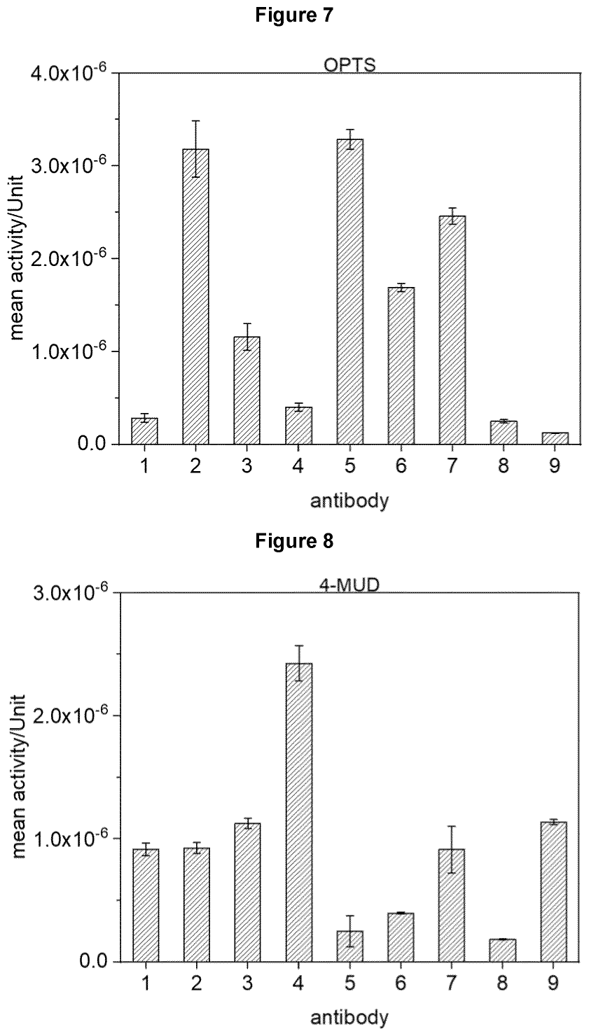

FIG. 7 shows the hydrolytic activities for the different antibody solutions following dialysis using the OPTS assay. A broad range of hydrolytic activity was observed, differing by more than one order of magnitude (from 1.24*10⁻⁷ units for antibody Ab9 to 3.28*10⁻⁶ units for Ab), which could be explained by the presence of different types or amounts of esterases in the protein solutions. The results shown in FIG. 7 were all determined at a pH of 5.5, corresponding to the pH of the solutions under investigation. Carboxylesterases typically show their maximum hydrolytic activity at about pH 6. It therefore seems that no carboxylesterase activity was detected in protein solutions 1, 4, 8 and 9.

The substrate OPTS (C8 chain length) has a chain length just in the transition zone between short- and long-chain fatty acid esters. It was therefore expected that although carboxylesterase activity is primarily measured, some lipase activity may also be detected.

To further characterize the detected activity, inhibition of hydrolytic activity was tested using the lipase inhibitor orlistat (10 μM), the protease inhibitor PMSF (1 mM) and the metal chelator EDTA (10 mM) in all 9 antibody samples. Inhibitors were added to the sample following dialysis prior to addition of the master mix freshly mixed with the substrate and measured for 30 min at intervals of 15 s. Neither orlistat, nor PMSF or EDTA significantly reduced the measured hydrolytic activity in any of the samples (data not shown).

### Example 8: Determination of Hydrolytic Activities in Protein Solutions Using the 4-MUD Assay

The data generated using the OPTS assay were compared with an already established micelle-containing assay using 4-MUD as substrate (FIG. 8), which was developed for detecting hydrolytic activity of lipases.

This 4-MUD assay was used to examine the same protein solution samples that were examined with the OPTS assay. Care was taken to keep the experimental conditions the same to allow a reliable comparison of the results, i.e., the same volume of antibody samples, measurements were taken at pH 5.5 and 25° C. and the kinetics were recorded for 30 min with a time interval of 15 s in BR measurement mode. The reaction mixtures only differed in substrate and substrate concentration (30 μM 4-MUD) and in the use of CHAPS (Roth, Deutschland) as detergent, as well as a different substrate-specific excitation wavelength λex and emission wavelength λem.

Results of kinetics determined using the 4-MUD assay are shown in FIG. 8. Again, a broad range of hydrolytic activity was observed, differing by more than one order of magnitude (1.8*10⁻⁷ units in protein solution 4 and 2.3*10⁻⁶ units in protein solution 8). Protein solutions 6, 7, 8 and 9 have a target concentration of polysorbate 20 (Serva, Germany) of 100 μg*mL-1. A qualitative comparison of the hydrolytic activities detected with OPTS or 4-MUD as substrate are shown in FIG. 9.

No clear trend is derivable from the data shown in FIG. 9, comparing the hydrolytic activity detected using the OPTS assay and the 4-MUD assay. Samples that show comparatively high activity in the OPTS assay (e.g. Ab5) show very low activity in the 4-MUD assay. In contrast, protein solution 4 shows high activity in the 4-MUD assay but only a very low hydrolytic activity in the OPTS assay. Thus, considering that the OPTS assay predominantly detects carboxylesterase activity and the 4-MUD assay predominantly detects lipase activity, carboxylesterase seem to be present mainly in samples 2, 5, 6 and 7 and lipases seem to be present mainly in samples 1, 4 and 9.

In sample Ab3, the hydrolytic activity detected using the OPTS assay and the 4-MUD assay were within the same range and in protein solution 8 hardly any hydrolytic activity was detected in any of the assays. Protein solution 5 shows the most distinct difference between the two substrates, showing hardly any hydrolytic activity using the 4-MUD assay, whereas the highest hydrolytic activity of all tested samples was detected using the OPTS assay. This indicates that hydrolytic activity of enzymes using hydrophilic substrates, such as carboxylesterases, seems to very strongly dominate that of lipases.

### Example 9: Influence of Inhibitors on Hydrolytic Activity in the 4-MUD Assay

The effect of the inhibitors orlistat (10 μM), PMSF (1 mM) and EDTA (10 mM) as previously tested in samples analysed using the OPTS assay were also tested in all 9 antibody samples using the 4-MUD assay. The results in FIG. 10 show the hydrolytic activity in the presence of the inhibitor relative to the same sample without the addition of an inhibitor (100%).

FIG. 10 shows that a pronounced inhibition of the hydrolytic activity by the lipase inhibitor orlistat (10 μM) took place in all 9 antibody samples examined. The resulting residual activities were in the range of 18-21% of the hydrolysis activity without addition of an inhibitor. For PMSF a sight inhibition of hydrolytic activity was observed in all protein solutions, except for antibody solution Ab1. The residual activities ranged from 72% in protein solution 6 to 28% in antibody solution Ab2. This indicates the observed hydrolytic activity in this sample was mainly due to an enzyme with a nucleophilic serine in the active site. The addition of EDTA, on the other hand, showed hardly any inhibition in any of the samples. It is therefore concluded that the hydrolytic activity detected in antibody solutions Ab1-Ab9 is not metal-dependent. The results of the different activities with the corresponding antibody solution in the OPTS and the 4-MUD assay are summarized in Table B below, wherein (++) means strong effect, (+) means weak effect and (−) means no significant effect.

TABLE B

| | Orlistat/PMSF/EDTA | Orlistat/PMSF/EDTA |
|---|---|---|
| Antibody solution | OPTS | 4-MUD |
| 1 | −/−/− | ++/−/− |
| 2 | −/−/− | ++/++/− |
| 3 | −/−/− | ++/+/− |
| 4 | −/−/− | ++/+/− |
| 5 | −/−/− | ++/++/− |
| 6 | −/−/− | ++/+/− |
| 7 | −/−/− | ++/+/− |
| 8 | −/−/− | ++/+/− |
| 9 | −/−/− | ++/+/− |

As may be taken from Table B, when OPTS was used as substrate, none of the inhibitors showed a significant effect on the detected hydrolytic activity. By contrast when 4-MUD was used as substrate the specific lipase inhibitor orlistat showed strong inhibition of the detected hydrolytic activity. Also, the serine protease inhibitor PMSF showed significant inhibition of hydrolytic activity detected with 4-MUD as substrate in antibody samples Ab2 and Ab5. The fact that the addition of EDTA showed no inhibition of hydrolytic activity in either the OPTS assay or the 4-MUD assay suggests that the hydrolytic enzymes present in the tested protein solution were not metalloenzymes.

The inhibitory effect of orlistat is due to the acylation of serine in the catalytic triad of the active site of the enzyme (Al-Suwailem, Al-Tamimi et al. 2006) and PMSF is also an inhibitor that binds to a serine in the active site (James 1978). Thus, these two inhibitors have in common that they render the active site less accessible for the substrates to be converted and therefore belong to the class of competitive inhibitors.

Further, the recently described allosteric inhibitor for lipases α-tropolone has been tested. Due to its good solubility in water this inhibitor could be suitable to further characterize the enzyme activity detected by the OPTS assay. However, tropolone at 10 μM turned out have a strong influence on both free fluorophores HPTS (detected in the OPTS assay) and 4-methylumbelliferone (4-MU) (detected in the 4-MUD assay), suggesting that the influence of tropolone on the hydrolytic activity described previously is most likely due to its influence on the fluorescence activity of the free fluorophore (fluorophore quenching) rather than an inhibitory effect.

### Example 10: Hydrolytic Activities of a Lipase Detected Using the OPTS Assay

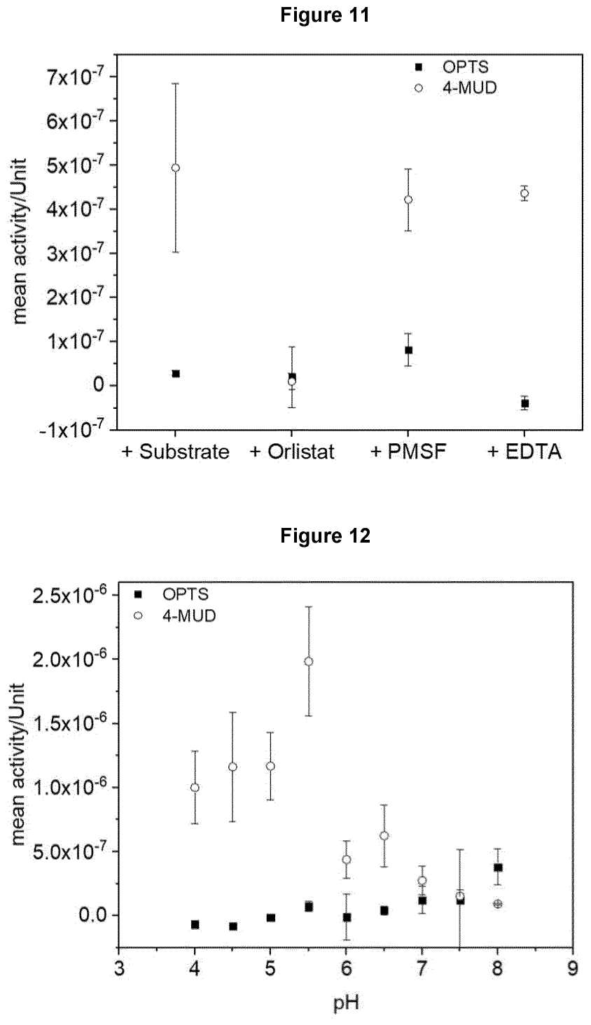

Further, a purified commercially available lipase with unknown substrate specificity was analysed using the 4-MUD and OPTS assay. First, hydrolysis of both soluble (OPTS) and hydrophobic (4-MUD) carboxyl esters, as well as the influence of orlistat, PMSF and EDTA on the hydrolytic activity of the lipase were investigated. The results are shown in FIG. 11.

The lipase generally showed significantly higher activities in the micelle-based 4-MUD assay compared to the OPTS assay. Only low to no activities were found with the OPTS assay. This indicates that polar, hydrophilic esters are no substrates for this hydrolase.

Further, no significant influence of the inhibitors on the hydrolytic activity was shown in the OPTS assay. For the hydrolytic activities detected using the 4-MUD substrate, orlistat almost completely inhibited the detected hydrolytic effectivity, while PMSF and EDTA had only minor effects. Since the tested lipase is not a serine protease, no inhibition was expected using the serine protease inhibitor PMSF.

Furthermore, the lipase was tested for its pH dependence in the OPTS and 4-MUD substrate assays. For this purpose, the hydrolytic activity was examined at pH=4-8. The results are shown in FIG. 12.

FIG. 12 shows that very low hydrolytic activity was detected using OPTS as substrate and slightly increased at pH 6.0 or higher. Using 4-MUD as substrate, the detected hydrolytic activity was higher at low pH with a maximum at pH 5.5. Furthermore, the detected activity strongly decreased at more basic pH. The lipase is a lysosomal lipase and hence the finding of a pH optimum at pH 5.5 is in agreement with a lysosomal pH at about pH 4.5 to 5.5.

The different results obtained using OPTS and 4-MUD as substrate are a further confirmation of the previous findings that the assays detect hydrolytic activities of different enzymes, such as carboxylesterases using OPTS and lipases using 4-MUD as substrate.

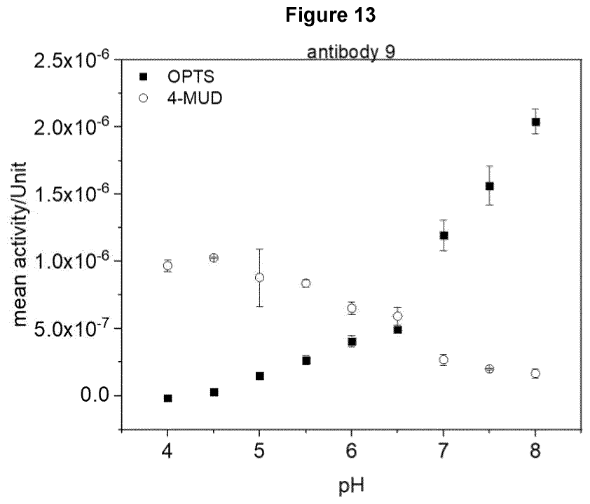

Comparing the hydrolytic activity detected using OPTS and 4-MUD as substrate, also with antibody solution Ab9 an increased activity was observed with increasing pH using OPTS as substrate and a decreasing activity was observed using 4-MUD as substrate (FIG. 13), with an overall stronger activity observed with the 4-MUD substrate. For antibody solution Ab9 there is a maximum activity in the acidic range, pH=4.5-5.

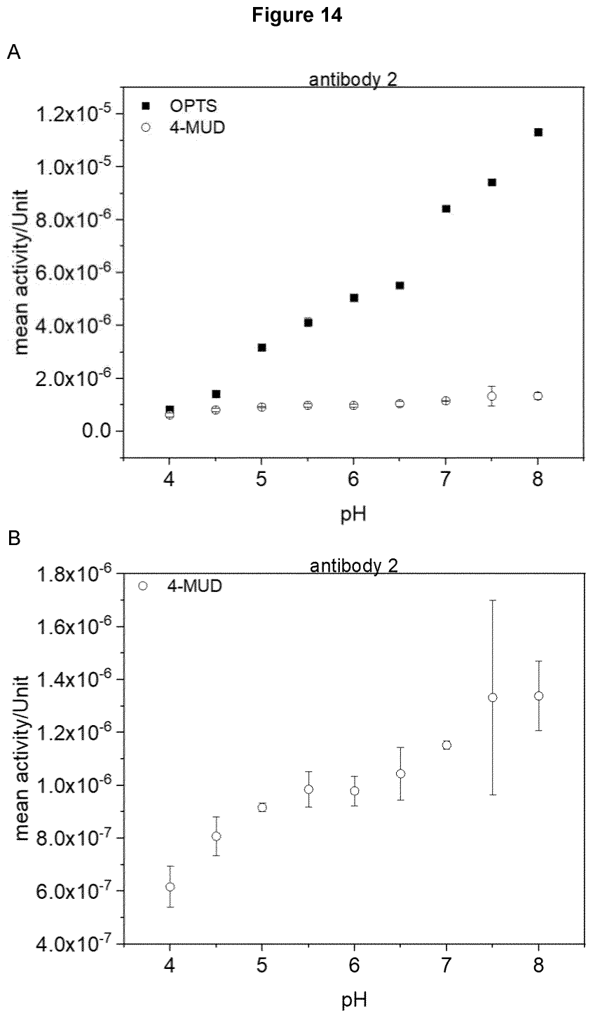

In contrast, antibody solution Ab2 showed a reverse behaviour, with low activity using the 4-MUD substrate and high activity using OPTS (FIG. 14A and B). The activities increase in both cases with increasing pH.

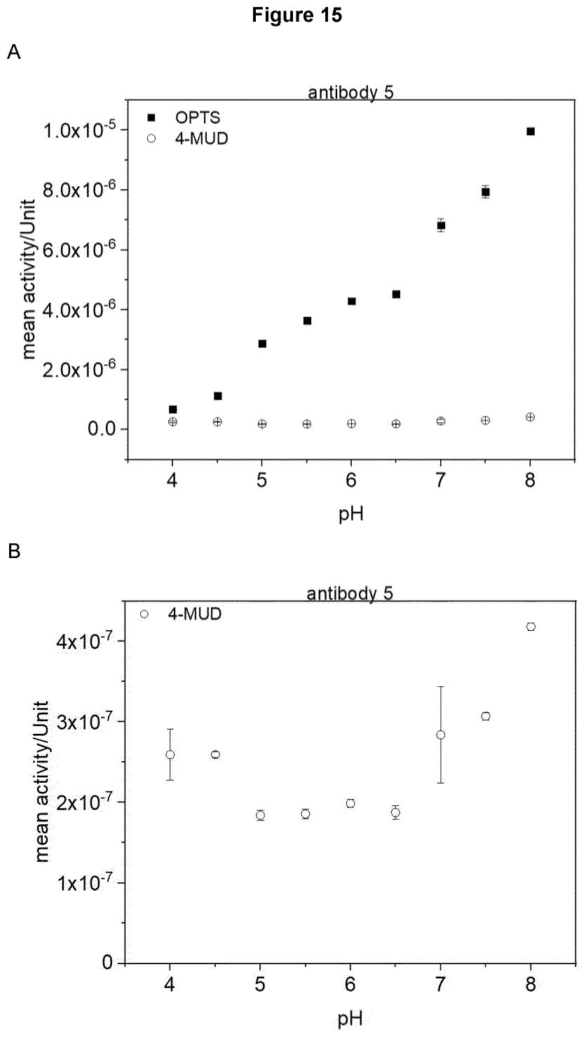

In FIG. 15, a similar behaviour is shown for antibody solution Ab5 with OPTS, but the activities are significantly lower with the 4-MUD substrate. Furthermore, the pH profile using 4-MUD are slightly different, showing a local maximum of activity in the acidic range (pH=4-4.5), a lower activity between pH 5 and 6.5 and increasing at higher pH. The pH profile measured with OPTS, however, shows a similar behaviour as for protein solution 2. The finding of almost no activity in the 4-MUD assay suggests that lipases are either absent or only present at very low concentrations in antibody solution Ab5 (and also antibody solutions Ab6 and Ab8; see FIG. 9).

Comparison of the pH profile of the tested commercial lipase (FIG. 12) and antibody solution Ab2 (FIG. 14) or antibody solution Ab5 (FIG. 15) shows no similarity. However, antibody solution Ab9 shows a similar profile for the data of the OPTS assay and the 4-MUD substrate assay at pH 4-8. This could be an indication for the presence of this lipase in antibody solution Ab9. Since the hydrolytic activities in the OPTS assay and the coumarin substrate assay are in the same order of magnitude, other enzymes seem to be present that also contribute to the hydrolytic activity.

1. A method for detecting carboxylesterase activity of contaminating host cell protein in a sample comprising a recombinant protein of interest produced in a eukaryotic cell comprising
(a) providing at least one sample comprising a recombinant protein of interest produced in a eukaryotic cell in cell culture and at least one contaminating host cell protein;
(b) contacting the at least one sample with a reaction solution (comprising a hydrophilic substrate) to form a reaction mixture, wherein the reaction solution comprises:
(i) a buffer having a pH of about pH 4 to about pH 8,
(ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and
(iii) optionally a non-buffering salt;
(c) incubating the sample and the substrate in the reaction mixture;
(d) detecting carboxylesterase activity of the at least one contaminating host cell protein by measuring hydrolysis of the substrate HPTS ester and detecting the fluorescence intensity of the released chromophore 1-hydroxypyrene-3,6,8-trisulfonic acid or a salt thereof (HPTS);
optionally measuring hydrolysis by detecting the fluorescence intensity of the released chromophore HPTS over time, while incubating the sample and the substrate in the reaction mixture according to step (c).

2. The method of claim 1, wherein
(a) the sample and the substrate in the reaction mixture are incubated for any time period between 2 min and 5 hours and/or
(b) multiple reaction mixtures are analysed in parallel; and/or
(c) wherein the reaction mixture has a volume of 300 μl or less.

3. The method of claim 1, wherein the fluorescence of the released chromophore HPTS is determined using an excitation wavelength within a range of 401-405 nm and an emission wavelength within a range of 510-516 nm.

4. The method of claim 1, wherein the substrate HPTS ester is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof.

5. The method of claim 1, wherein the method further comprises
(bi) contacting the at least one sample comprising a recombinant protein of interest produced in a eukaryotic cell in cell culture and at least one contaminating host cell protein of step (a) in a separate reaction set-up with a reaction solution comprising a lipophilic substrate to form a reaction mixture, wherein the reaction solution comprises:
(i) a buffer having a pH of about pH 4 to about pH 8,
(ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester,
(iii) optionally a non-buffering salt, and
(iv) a non-denaturing detergent not having an ester-bond, wherein the detergent is a non-ionic or zwitterionic detergent,
(ci) incubating the sample and the substrate in the reaction mixture of step (bi); and
(di) detecting lipase activity of the at least one contaminating host cell protein by measuring hydrolysis of the substrate 4-MU ester and detecting the fluorescence intensity of the released chromophore 4-MU;
optionally measuring hydrolysis by detecting the fluorescence intensity of the released chromophore 4-MU over time, while incubating the sample and the substrate in the reaction mixture according to step (ci).

6. The method of claim 5, wherein
(a) the lipophilic substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate;
(b) the detergent has a final concentration in the reaction mixture above its critical micelle concentration in the reaction mixture; and/or
(c) the detergent
(i) is selected from the group consisting of CHAPS, CHAPSO and Zwittergent; or
(ii) is CHAPS and is provided at a final concentration in the reaction mixture of about 8 mM to about 20 mM; or
(iii) is not polyethylene glycol tert-octylphenyl ether (Triton X-100) and not polyethylene glycol nonylphenyl ether (NP-40).

7. The method of claim 1, wherein the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino)pro-panesulfonic acid), Tricine (N-tris(hydroxymethyl)methylglycine), Na₂HPO₄ and NaH₂PO₄.

8. The method of claim 1, wherein the buffer
(a) has a pH of about 5 to about 7.5; and/or
(b) is a multi-component buffer having a buffering range from at least about pH 4 to at least about pH 8.

9. The method of claim 1, wherein
(a) the non-buffering salt is selected from the group consisting of NaCl, KCl and CaCl₂; and/or
(b) the non-buffering salt has a concentration of about 100 mM to about 200 mM in the reaction mixture; and/or
(c) the ionic strength of non-buffering salt is about 200 mM or less in the reaction mixture; and/or
(d) the cumulative ionic strength of the buffer and the non-buffering salt in the reaction mixture is about 450 mM or less in the reaction mixture.

10. The method of claim 1, wherein
(a) the at least one sample is a harvested cell culture fluid (HCCF), an in-process control (IPC) sample, a UF/DF filtrate, a drug substance sample or a drug product sample;
(b) the recombinant protein of interest is produced in a CHO cell and the at least one contaminating host cell protein is a CHO host cell protein (CHOP);
(c) the recombinant protein of interest is not a carboxylesterase or a lipase and/or an enzyme having carboxylesterase or lipase activity; and/or
(d) the recombinant protein of interest is selected from the group consisting of an antibody, an antibody fragment, an antibody derived molecule and a fusion protein.

11. A method of manufacturing a recombinant protein of interest comprising the steps of
(i) cultivating a eukaryotic cell expressing a recombinant protein of interest in cell culture;
(ii) harvesting the recombinant protein of interest;
(iii) purifying the recombinant protein of interest; and
(iv) optionally formulating the recombinant protein of interest into a pharmaceutically acceptable formulation suitable for administration; and
(v) obtaining at least one sample comprising the recombinant protein of interest in steps (ii), (iii) and/or (iv);
wherein the method further comprises detecting carboxylesterase activity in a sample comprising the recombinant protein of interest and at least one contaminating host cell protein comprising:
(a) providing the at least one sample obtained in step (v) comprising the recombinant protein of interest produced in a eukaryotic cell in cell culture and at least one contaminating host cell protein;
(b) contacting the at least one sample with a reaction solution comprising a hydrophilic substrate to form a reaction mixture, wherein the reaction solution comprises:
(i) a buffer having a pH of about pH 4 to about pH 8,
(ii) a hydrophilic substrate, wherein the substrate is a saturated unbranched-chain fatty acid (C6-C12) ester of 1-hydroxypyrene-3,6,8-trisulfonic acid or a salt thereof (substrate HPTS ester), and
(iv) optionally a non-buffering salt;
(c) incubating the sample and the substrate in the reaction mixture; and
(d) detecting carboxylesterase activity of the at least one contaminating host cell protein by measuring hydrolysis of the substrate HPTS ester and detecting the fluorescence intensity of the released chromophore HPTS;
wherein the method optionally further comprises detecting lipase activity in a sample comprising
(bi) contacting the at least one sample comprising a recombinant protein of interest produced in a eukaryotic cell and at least one contaminating host cell protein of step (a) in a separate reaction set-up with a reaction solution comprising a lipophilic substrate to form a reaction mixture, wherein the reaction solution comprises;
(i) a buffer having a pH of about pH 4 to about pH 8,
(ii) a lipophilic substrate comprising the chromophore 4-methylumbelliferyl (4-MU) in the form of a 4-MU ester, wherein the 4-MU ester is a saturated unbranched-chain fatty acid (C6-C16) 4-MU ester,
(iii) optionally a non-buffering salt, and
(iv) a non-denaturing detergent not having an ester-bond, wherein the detergent is a non-ionic or zwitterionic detergent,
(ci) incubating the sample and the substrate in the reaction mixture; and
(di) detecting lipase activity of the at least one contaminating host cell protein by measuring hydrolysis of the 4-MU ester and detecting the fluorescence intensity of the released chromophore 4-MU;
optionally measuring hydrolysis by detecting the fluorescence intensity of the released chromophore HPTS and/or 4-MU over time, while incubating the sample and the substrate in the reaction mixture according to step (c) or (ci), respectively.

12. The method of claim 11, comprising obtaining at least one sample comprising the recombinant protein of interest in
step (ii), wherein the sample is a harvested cell culture fluid (HCCF) or a cell lysate;
step (iii), wherein the sample is an in-process control (IPC) sample; and/or
step (iv), wherein the sample is a UF/DF sample, a drug substance sample or a drug product sample;
optionally comprising obtaining at least one sample comprising the recombinant protein of interest produced in a eukaryotic cell in cell culture and at least one contaminating host cell protein in step (iii), comprising obtaining at least one sample before and after affinity chromatography, before and after acid treatment, before and after depth filtration, and/or before and after ion exchange chromatography.

13. A kit for determining contaminating carboxylesterase and/or lipase activity in a sample comprising a recombinant protein of interest comprising:
(i) a buffer having a pH of about pH 4 to about pH 8;
(ii) a hydrophilic substrate and a lipophilic substrate, wherein
(a) the hydrophilic substrate is a saturated unbranched-chain fatty acid (C6-C12) ester of 1-hydroxypyrene-3,6,8-trisulfonic acid or a salt thereof (substrate HPTS ester); and
(b) the lipophilic substrate comprises the chromophore 4-methylumbelliferyl (4-MU) in the form of a 4-MU ester, wherein the 4-MU ester is a saturated unbranched-chain fatty acid (C6-C16) 4-MU ester (substrate 4-MU ester); and optionally
(iii) a non-buffering salt; and/or
(iv) a non-denaturing detergent not having an ester-bond, wherein the detergent is a non-ionic or zwitter-ionic detergent.

14. The kit of claim 13, wherein
(a) the hydrophilic substrate HPTS ester is selected from the group consisting of 1-octanoyloxy-pyrene-3,6,8-trisulfonic acid or a salt thereof (OPTS), 1-nonaoyloxy-pyrene-3,6,8-trisulfonic acid or a salt thereof and 1-decanoyloxy-pyrene-3,6,8-trisulfonic acid or a salt thereof; and/or
(b) the lipophilic substrate 4-MU ester is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate.

15. The kit of claim 13, wherein the kit further comprises one or more microtiter plate having 96 wells or a multiple of 96 wells.

16. A method for detecting in an assay carboxylesterase and lipase activity of at least one contaminating host cell protein in a sample comprising a recombinant protein of interest produced in a eukaryotic cell in cell culture, said method comprising utilizing a hydrophilic substrate HPTS ester and a lipophilic substrate 4-MU ester as a substrate,
wherein the hydrophilic substrate is a saturated unbranched-chain fatty acid (C6-C12) ester of 1-hydroxypyrene-3,6,8-trisulfonic acid or a salt thereof (substrate HPTS ester), and
wherein the lipophilic substrate is a saturated unbranched-chain fatty acid (C6-C16) 4-MU ester.

17. A method for detecting in an assay carboxylesterase activity of at least one contaminating host cell protein in a sample comprising a recombinant protein of interest produced in a eukaryotic cell in cell culture, said method comprising utilizing a hydrophilic substrate HPTS ester as a substrate,
wherein the hydrophilic substrate is a saturated unbranched-chain fatty acid (C6-C12) ester of 1-hydroxypyrene-3,6,8-trisulfonic acid or a salt thereof (substrate HPTS ester).

18. The method of claim 16, wherein the recombinant protein is produced in a CHO cell and the at least one contaminating host cell protein is a CHO host cell protein (CHOP).

19. The method of claim 17, wherein the recombinant protein is produced in a CHO cell and the at least one contaminating host cell protein is a CHO host cell protein (CHOP).
